(19) United States
(12) Patent Application Publication
(10) Pub. No.: US 2026/0308660 A1
(43) Pub. Date: Oct. 8, 2026

(54) METHOD FOR ANALYZING ACTIVITY OF NUCLEIC ACID POLYMERASE USING SELF-DIMER PRIMER

(71) Applicant: SEEGENE, INC., Seoul (KR)

(72) Inventors: Hyobeen LEE, Seoul (KR); Byungjun AHN, Seoul (KR); Myung-Il KIM, Seoul (KR); Jaemin LEE, Seoul (KR)

(73) Assignee: SEEGENE, INC., Seoul (KR)

(21) Appl. No.: 19/681,864

(22) Filed: May 19, 2026

Related U.S. Application Data
(63) Continuation of application No. PCT/KR2024/018906, filed on Nov. 26, 2024.

(30) Foreign Application Priority Data
Nov. 27, 2023 (KR) 10-2023-0166983

(51) Int. Cl. C12Q 1/48; C12Q 1/6816; C12Q 1/686; C12Q 1/6876

(52) U.S. Cl. CPC C12Q 1/48; C12Q 1/6816; C12Q 1/686; C12Q 1/6876; C12Y 207/07

(57) ABSTRACT

A method for analyzing the activity of a nucleic acid polymerase of interest using a self-dimer primer is disclosed. The method requires fewer factors to be considered in design and thus are easy to design, compared to conventional methods using both primers and templates. In addition, the method is simple, fast, cost-effective, safe, and user-friendly compared to conventional methods using radioisotopes.

### CROSS-REFERENCE TO RELATED APPLICATIONS

This application is a bypass continuation application of PCT Application No. PCT/KR2024/018906 filed on Nov. 26, 2024, which claims priority from Korean Patent Application No. 10-2023-0166983, filed on Nov. 27, 2023, in the Korean Intellectual Property Office, the disclosures of which are incorporated herein by reference in their entireties.

### INCORPORATION BY REFERENCE OF SEQUENCE LISTING

The instant application contains a Sequence Listing which has been filed electronically in xml format and is hereby incorporated by reference in its entirety. Said xml copy, created on Apr. 5, 2026, is named Q319782SEQST26.xml and is 4,670 bytes in size.

### TECHNICAL FIELD

The present disclosure relates to a method for analyzing the activity of a nucleic acid polymerase by using a self-dimer primer having a unique structure and a composition for analyzing the activity of a nucleic acid polymerase, comprising the self-dimer primer.

### BACKGROUND ART

Molecular diagnosis is one of the in vitro diagnostic testing methods that amplifies DNA or RNA, e.g., using a polymerase chain reaction (PCR) and analyzes the amplified products. Recently, PCR has become important as a molecular diagnostic test for preemptive response to COVID-19 pandemic. Nucleic acid polymerases that are stable at a wide range of temperatures and exhibit high activity is required to enable this PCR.

Many molecular diagnostic reagents for target nucleic acid amplification include polymerases derived from thermophilic microorganisms (extremophiles) that are stable at high temperatures, a representative example of which is Taq DNA polymerase from T. aquaticus (Chien A., Edgar D. B., Trela J. M. J. Bacteriol. 1976, 127, 1550-1557).

DNA polymerases are a group of polymerases that catalyze the synthesis of DNA using DNA or RNA as a template. Since these polymerases performs a polymerization reaction by consuming nucleoside triphosphates (dNTPs) from a primer bound to a template as a substrate, their activities are measured by how fast the substrate is consumed, which is defined as a unit of the polymerases.

A unit of polymerase is generally defined as the amount of polymerase that incorporates a unit nucleoside triphosphate into acid insoluble form within a unit time at a unit temperature. For example, according to New England Biolabs (NEB), one unit of Taq DNA polymerase is defined as the amount of polymerase that will incorporate 15 nmol of dNTP into acid insoluble form in 30 minutes at 75° C., and one unit of Bst DNA polymerase, Large Fragment, is defined as the amount of polymerase that will incorporate 10 nmol of dNTP into acid insoluble form in 30 minutes at 65° C.

The definitions of these units vary slightly depending on the manufacturer or product, the temperature, the amount of substrate, and the type of material containing radioisotope, but the unit is typically determined by measuring the amount of a substrate consumed using radioisotopes or by measuring the amount of an insoluble polymer to calculate the reaction rate (Charles C. Richardson, Carl L. Schildkraut, H. Vasken Aposhian, Arthur Kornberg J. Biol. Chem. 1964, 239(1), 222-232).

Despite its high accuracy, the method of measuring the unit of polymerase as above has environmental and health problems due to the use of radioisotopes. Thus, attempts have been made for decades to define the unit of polymerase without radioisotopes. For example, measurement of fluorescence generated by DNA polymerase-mediated release of single-stranded binding protein (Mark A. Griep, Anal. Biochem. 1995, 232, 180-189), measurement of binding of PicoGreen™ to double-stranded DNA (Seville, M., West A. B., Cull, M. G. and McHenry, C. S. Biotechniques 21, 1996, 664, 668, 670, 672; Tveit, H. and Kristensen, T. Anal. Biochem. 2001, 289, 96-98), microplate coupling and detection of fluorescently-labeled nucleotides (Yu, Liming, Hu, G. and Howells, L. Biotechniques 33, 2002, 938-941), molecular beacon based method (Ma, C, Tang, Z., Wang, K., Tan, W. Li, J. Li, W. W. Yang, Z. and Liu, L. Anal. Biochem. 2006, 353, 141-143), electrochemical method (Luo, X. and Hsing I. Electroanalysis 23, 2011, 923-926), or method of applying additional kinetic techniques (Jesse L. Montgomery, Nick Rejali, Carl T. Wittwer, Anal. Biochem. 2013, 441(2), 133-139) have been developed.

The methods as illustrated above measure the efficiency of unidirectional polymerization of primers by a polymerase along a single-stranded template such as M13mp18. The polymerization efficiency is defined by the amount of dNTP consumed in a unit time at the optimum temperature for the polymerase. However, these methods have disadvantages such as difficulty in optimizing reaction conditions and sequences of primers and probes used, non-specific interference, and low reaction sensitivity.

Therefore, in order to address these disadvantages, there remains a need to develop a novel method for analyzing the activity of a nucleic acid polymerase.

Throughout this application, various patents and publications are referenced and citations are provided in parentheses. The disclosure of these patents and publications in their entirety are hereby incorporated by references into this application in order to more fully describe this invention and the state of the art to which this invention pertains.

### DISCLOSURE

### Technical Problem

The present inventors have sought to develop a method for analyzing the activity of a nucleic acid polymerase used in the diagnosis field, particularly nucleic acid amplification reactions such as real-time PCR. As a result, the present inventors have found that the activity of a nucleic acid polymerase of interest can be conveniently determined using a self-dimer primer without any additional oligonucleotide or template.

Accordingly, it is an object of the present disclosure to provide a method for analyzing the activity of a nucleic acid polymerase of interest.

It is another object of the present disclosure to provide a composition for analyzing the activity of a nucleic acid polymerase of interest.

It is still another object of the present disclosure to provide a self-dimer primer.

Other objects and advantages of the present invention will become apparent from the detailed description to follow taken in conjugation with the appended claims and drawings.

### Technical Solution

In an aspect, there is provided a method for analyzing the activity of a nucleic acid polymerase of interest, comprising the steps of: (a) incubating the nucleic acid polymerase of interest with a composition for analyzing activity comprising: (i) a self-dimer primer, (ii) an intercalating dye, and (iii) dNTPs or NTPs, wherein the self-dimer primer comprises, in the 5′ to 3′ direction, a 5′-hairpin-non-forming portion and a 3′-hairpin-forming portion, wherein during said incubating, the self-dimer primer hybridizes with another self-dimer primer between 3′-hairpin-forming portions to form a self-dimer, and both strands of the resulting self-dimer are extended by the activity of the nucleic acid polymerase of interest to form an extended dimer, wherein the formation of the extended dimer provides a detectable signal from the intercalating dye; and (b) measuring the detectable signal over time, wherein the change in signal over time is indicative of the activity of the nucleic acid polymerase of interest.

In certain embodiments, the nucleic acid polymerase of interest is a DNA polymerase.

In certain embodiments, the DNA polymerase is derived from a bacterium selected from the group consisting of Thermus aquaticus, Thermus thermophilus, Thermus filiformis, Thermus flavus, Thermococcus litoralis, Thermus antranikianii, Thermus caldophilus, Thermus chliarophilus, Thermus igniterrae, Thermus lacteus, Thermus oshimai, Thermus ruber, Thermus rubens, Thermus scotoductus, Thermus silvanus, Thermus species Z05, Thermus species sps 17, Thermotoga maritima, Thermotoga neapolitana, Thermosipho africanus, Thermococcus barossii, Thermococcus gorgonarius, Pyrococcus furiosus, Pyrococcus woesei, Pyrococcus horikoshii, Pyrococcus abyssi, Pyrodictium occultum, Aquifex pyrophilus, and Aquifex aeolicus.

In certain embodiments, the 5′-hairpin-non-forming portion of the self-dimer primer has a nucleotide sequence substantially non-complementary to the 5′-hairpin-non-forming portion and the 3′-hairpin-forming portion of another self-dimer primer.

In certain embodiments, the 5′-hairpin-non-forming portion of the self-dimer primer is 5 to 50 nucleotides in length.

In certain embodiments, the 3′-hairpin-forming portion of the self-dimer primer has a nucleotide sequence substantially complementary to the 3′-hairpin-forming portion of another self-dimer primer.

In certain embodiments, the 3′-hairpin-forming portion of the self-dimer primer has a substantially palindromic sequence.

In certain embodiments, when the 3′-hairpin-forming portion of the self-dimer primer is n nucleotides in length, the m^{th} nucleotide base from the 3′-terminus is complementary to the n−m+1^{th} nucleotide base, wherein n is an integer from 5 to 50, and m is an integer of 1 or greater.

In certain embodiments, the 3′-hairpin-forming portion of the self-dimer primer is 5 to 50 nucleotides in length.

In certain embodiments, the ratio of the length of the 5′-hairpin-non-forming portion to the length of the 3′-hairpin-forming portion of the self-dimer primer is about 1:1.

In certain embodiments, the self-dimer primer is 10 to 100 nucleotides in length.

In certain embodiments, the intercalating dye is selected from the group consisting of EvaGreen, ethidium bromide, SYBR Green I, SYBR Gold, Yo-Yo, Yo-Pro, TOTO, SYTO9, BEBO, BOXTO, Hoechst dyes, LC Green Plus, ResoLight, Chromofy, and PicoGreen.

In certain embodiments, the incubating is performed under isothermal conditions such that the nucleic acid polymerase of interest functions and the self-dimer primer does not form a hairpin dimer by intra-strand hybridization.

In certain embodiments, the isothermal condition is at a temperature selected from 65 to 80° C.

In certain embodiments, the composition for analyzing activity does not comprise any additional oligonucleotide other than the self-dimer primer.

In certain embodiments, the change in signal is measured over a period of time during which a plot of signal intensity versus incubation time represents a linear function.

In certain embodiments, the change in signal over time is converted into an amount of dNTP consumed in a unit time of incubation, thereby indicating the activity of the nucleic acid polymerase of interest.

In another aspect, there is provided a composition for analyzing the activity of a nucleic acid polymerase of interest, comprising a self-dimer primer, wherein the self-dimer primer comprises, in the 5′ to 3′ direction, a 5′-hairpin-non-forming portion and a 3′-hairpin-forming portion, wherein during said incubating, the self-dimer primer hybridizes with another self-dimer primer between 3′-hairpin-forming portions to form a self-dimer, and both strands of the resulting self-dimer are extended by the activity of the nucleic acid polymerase of interest to form an extended dimer, wherein the formation of the extended dimer provides a detectable signal from the intercalating dye, wherein the change in signal over time is indicative of the activity of the nucleic acid polymerase.

In certain embodiments, the composition further comprises an intercalating dye.

In certain embodiments, the composition further comprises dNTPs or NTPs.

In another aspect, there is provided a self-dimer primer comprising, in the 5′ to 3′ direction, a 5′-hairpin-non-forming portion and a 3′-hairpin-forming portion, wherein the 5′-hairpin-non-forming portion has a nucleotide sequence substantially non-complementary to the 5′-hairpin-non-forming portion and the 3′-hairpin-forming portion of another self-dimer primer, and the 3′-hairpin-forming portion has a nucleotide sequence substantially complementary to the 3′-hairpin-forming portion of another self-dimer primer, wherein the self-dimer primer is capable of hybridizing with another self-dimer primer between 3′-hairpin-forming portions to form a self-dimer.

### Advantageous Effects

The features and advantages of the present disclosure are summarized as follows:
(a) Since the method of the present disclosure uses a single type of self-dimer primer, it requires fewer factors to be considered in design and thus are easy to design, compared to conventional methods using both primers and templates. In addition, the conventional methods described above have disadvantages of inconsistent length of the final product, low signal consistency, and inaccurate analysis result due to the formation of unwanted dimers, whereas the method of the present disclosure can overcome these problems.
(b) The method of the present disclosure is simple, fast, cost-effective, safe, and user-friendly compared to conventional methods using radioisotopes.
(c) The method of the present disclosure can be applied to analyze the activity and purity and monitor quality control for various nucleic acid polymerases in their production. Further, it can be used in various places such as large hospitals, clinical laboratories, and research institutes to perform diagnosis of diseases using nucleic acid polymerases.

### DESCRIPTION OF DRAWINGS

FIGS. 1A-1C illustrate the structure of a self-dimer primer according to the present disclosure. FIG. 1A shows the structure of a single-stranded self-dimer primer that may be present prior to incubating according to the method of the present disclosure, wherein the 5′-hairpin-non-forming portion of the self-dimer primer is represented by a solid black line, and the 3′-hairpin-forming portion of the self-dimer primer is represented by a solid gray line. FIG. 1B shows the structure of a hairpin dimer formed by intra-strand hybridization, which may be present prior to incubating according to the method of the present disclosure, wherein the 3′-hairpin-forming portion of the self-dimer primer forms a hairpin dimer by intra-strand hybridization under certain temperature conditions. FIG. 1C shows the structure of a self-dimer formed by inter-strand hybridization, which may be present before or during incubating according to the method of the present disclosure, wherein the 3′-hairpin-forming portions of the two self-dimer primers hybridize to each other under certain temperature conditions to form a self-dimer.

FIG. 2 illustrates a process in which a signal is generated by self-dimer primers. During incubating according to the method of the present disclosure, the 3′-hairpin-forming portions of the two self-dimer primers hybridize to each other to form a self-dimer (top). Then, both strands of the resulting self-dimer are extended by the activity of the nucleic acid polymerase to form an extended dimer, and the formation of the extended dimer provides a detectable signal from the intercalating dye (bottom).

FIG. 3 shows the structure of Self-Dimer Primer #1 used in the Example. Self-Dimer Primer #1 consists of a 5′-hairpin-non-forming portion of 28 nucleotides in length and a 3′-hairpin-forming portion of 32 nucleotides in length. Self-Dimer Primer #1 can form a self-dimer (top) or a hairpin dimer (bottom).

FIG. 4 is a plot of fluorescence signal versus time, which is obtained by analyzing the activity of Taq DNA polymerase at various concentrations using Self-Dimer Primer #1.

FIG. 5 is a plot of consumption of dNTP versus concentration of Taq DNA polymerase, which is obtained by analyzing the activity of Taq DNA polymerase at various concentrations using Self-Dimer Primer #1.

FIG. 6 shows the structure of Self-Dimer Primer #2 used in the Example. Self-Dimer Primer #2 consists of a 5′-hairpin-non-forming portion of 8 nucleotides in length and a 3′-hairpin-forming portion of 32 nucleotides in length. Self-Dimer Primer #2 can form a self-dimer (top) or a hairpin dimer (bottom).

FIG. 7 shows the structure of Self-Dimer Primer #3 used in the example. Self-Dimer Primer #3 consists of a 5′-hairpin-non-forming portion of 16 nucleotides in length and a 3′-hairpin-forming portion of 32 nucleotides in length. Self-Dimer Primer #3 can form a self-dimer (top) or a hairpin dimer (bottom).

FIG. 8 shows the structure of Self-Dimer Primer #4 used in the Example. Self-Dimer Primer #4 consists of a 5′-hairpin-non-forming portion of 16 nucleotides in length and a 3′-hairpin-forming portion of 32 nucleotides in length. Self-Dimer Primer #4 can form a self-dimer (top) or a hairpin dimer (bottom).

FIGS. 9A-9C are plots of fluorescence signal versus time, which are obtained by analyzing the activity of Taq DNA polymerase at various concentrations using Self-Dimer Primer #2 (FIG. 9A), Self-Dimer Primer #3 (FIG. 9B), and Self-Dimer Primer #4 (FIG. 9C).

FIGS. 10A-10D are plots of fluorescence signal versus time, which are obtained by analyzing the activity of Taq DNA polymerase (available from Roche; FIG. 10A), rTth DNA polymerase (available from TOYOBO; FIG. 10B), Bst DNA polymerase, large fragment (available from NEB; FIG. 10C), and Pfu DNA polymerase (available from PROMEGA; FIG. 10D) using Self-Dimer Primer #1.

FIGS. 11A-11D are plots of consumption of dNTP versus concentration of polymerase, which are obtained by analyzing the activity of Taq DNA polymerase (available from Roche; FIG. 11A), rTth DNA polymerase (available from TOYOBO; FIG. 11B), Bst DNA polymerase, large fragment (available from NEB; FIG. 11C), and Pfu DNA polymerase (available from PROMEGA; FIG. 11D) using Self-Dimer Primer #1.

### BEST MODE

The present inventors have sought to develop a method for analyzing the activity of a nucleic acid polymerase used in the diagnosis field, particularly nucleic acid amplification reactions such as real-time PCR. As a result, the present inventors have found that the activity of a nucleic acid polymerase of interest can be conveniently determined using a self-dimer primer without any additional oligonucleotide or template.

One of the features of the present invention lies in analyzing the activity of a nucleic acid polymerase of interest by forming a self-dimer using self-dimer primers having a unique structure and extending the self-dimer by the nucleic acid polymerase of interest.

### I. Method for Analyzing the Activity of a Nucleic Acid Polymerase of Interest

In an aspect, there is provided a method for analyzing the activity of a nucleic acid polymerase of interest, comprising the steps of.
(a) incubating the nucleic acid polymerase of interest with a composition for analyzing activity comprising:
   (i) a self-dimer primer,
   (ii) an intercalating dye, and
   (iii) dNTPs or NTPs,
wherein the self-dimer primer comprises, in the 5′ to 3′ direction, a 5′-hairpin-non-forming portion and a 3′-hairpin-forming portion,
wherein during said incubating, the self-dimer primer hybridizes with another self-dimer primer between 3′-hairpin-forming portions to form a self-dimer, and both strands of the resulting self-dimer are extended by the activity of the nucleic acid polymerase of interest to form an extended dimer,
wherein the formation of the extended dimer provides a detectable signal from the intercalating dye; and
(b) measuring the detectable signal over time, wherein the change in signal over time is indicative of the activity of the nucleic acid polymerase.

The method of the present disclosure uses a self-dimer primer having a unique structure and an intercalating dye that binds to a double-stranded self-dimer made from two self-dimer primers to generate a fluorescence signal without radioisotopes to determine the activity, i.e., the unit of a nucleic acid polymerase of interest.

In particular, the method of the present disclosure allows for determination of the activity, i.e., the unit of a nucleic acid polymerase of interest using self-dimer primers designed to form a self-dimer, without a template DNA or RNA which is commonly utilized in a nucleic acid amplification reaction such as PCR.

Specifically, the method of the present disclosure allows two self-dimer primers to form a self-dimer by inter-strand hybridization between 3′-hairpin-forming portions and then both strands of the self-dimer to be extended by a nucleic acid polymerase of interest, wherein the consumption of dNTP in a unit time at a certain temperature is estimated, and the estimated consumption of dNTP indicates the activity of the nucleic acid polymerase of interest. In this method, an intercalating dye that binds to double-stranded DNA (referred to herein as an extended dimer) to generate a fluorescence signal is used to indicate the amount of the extended dimer polymerized by the nucleic acid polymerase, and the consumption of dNTP is estimated by the generated fluorescence signal.

As used herein, the term “activity” in the context of a nucleic acid polymerase refers to the polymerization activity or ability of a nucleic acid polymerase.

As used herein, the term “analyzing the activity,” “measuring the activity,” or “determining the activity” in the context of a nucleic acid polymerase refers to determining how many dNTP are consumed by a unit mass or unit dilution of the nucleic acid polymerase at a given temperature for a given time.

Specifically, the term means adding a nucleic acid polymerase at various concentrations to a self-dimer formed by self-dimer primers of the present disclosure and measuring the polymerization rate of the nucleic acid polymerase at the optimum temperature.

Hereinafter, the method of the present disclosure will be described in detail.

Step (a): Incubation of a Nucleic Acid Polymerase of Interest with a Composition for Analyzing Activity

In step (a), a nucleic acid polymerase of interest is incubated with a composition for analyzing activity comprising: (i) a self-dimer primer, (ii) an intercalating dye, and (iii) dNTPs or NTPs.

### Nucleic Acid Polymerase

As used herein, the term “nucleic acid polymerase of interest” refers to a nucleic acid polymerase to be analyzed for activity by the method of the present disclosure. The term encompasses nucleic acid polymerases having unknown activity as well as nucleic acid polymerases having known activity requiring confirmation or verification.

The nucleic acid polymerase of interest may be a final or intermediate product which is obtained after or during its production and/or purification. For example, where a nucleic acid polymerase is expressed in a host cell, the nucleic acid polymerase may be an intermediate product during culture of a transformed host cell, expression of the nucleic acid polymerase from the host cell, or purification of the expressed nucleic acid polymerase.

As used herein, the term “nucleic acid polymerase” refers to an enzyme that synthesizes the long chain of a nucleic acid. The nucleic acid polymerase includes DNA polymerases and RNA polymerases that are used to assemble DNA and RNA molecules, respectively, by copying a DNA template strand using base-pairing interactions or RNA by half ladder replication.

In certain embodiments, the nucleic acid polymerase of interest is a DNA polymerase. In certain embodiments, the nucleic acid polymerase of interest is a template-dependent DNA polymerase.

Examples of the DNA polymerase include, but not limited to, Taq, Tth, Pfu, Bst Full length, Bst Large fragment, Bst 2.0, Bst 3.0, Vent, Vent (exo-), Deep Vent, Deep Vent (exo-), Bca, Bsu, Klenow fragment, Klenow fragment (exo-), Φ29, KOD, GspSSD, GspF, OmniAmp polymerase, and SD polymerase.

Particularly, the DNA polymerase is derived from a bacterium selected from the group consisting of Thermus aquaticus, Thermus thermophilus, Thermus filiformis, Thermus flavus, Thermococcus litoralis, Thermus antranikianii, Thermus caldophilus, Thermus chliarophilus, Thermus igniterrae, Thermus lacteus, Thermus oshimai, Thermus ruber, Thermus rubens, Thermus scotoductus, Thermus silvanus, Thermus species Z05, Thermus species sps 17, Thermotoga maritima, Thermotoga neapolitana, Thermosipho africanus, Thermococcus barossii, Thermococcus gorgonarius, Pyrococcus furiosus, Pyrococcus woesei, Pyrococcus horikoshii, Pyrococcus abyssi, Pyrodictium occultum, Aquifex pyrophilus, and Aquifex aeolicus.

In one embodiment, the nucleic acid polymerase of interest is a reverse transcriptase. The reverse transcriptase is an RNA-dependent DNA polymerase that catalyzes DNA synthesis using RNA as a template. Various types of reverse transcriptases known in the art can be used in the method of the present disclosure.

### Composition for Analyzing Activity

As used herein, the term “composition for analyzing activity” refers to a composition used to measure or determine the activity, i.e., polymerization activity or unit, of a nucleic acid polymerase of interest. The composition for analyzing activity may be used interchangeably with a reagent for analyzing activity or a reagent for unit measurement.

The composition for analyzing activity as used herein refers to a mixture of chemical components involved in the formation of a self-dimer, its extension, and thus the generation of a detectable signal, except for the nucleic acid polymerase whose activity is to be analyzed. The term is intended to exclude any physical or mechanical component such as containers, incubators, detectors, and the like.

The composition for analyzing activity according to the present disclosure comprises:
(i) a self-dimer primer,
(ii) an intercalating dye, and
(iii) dNTPs or NTPs.

### (i) Self-Dimer Primer

A key feature of the method of the present disclosure is the use of a self-dimer primer having a unique structure.

As used herein, the term “primer” refers to an oligonucleotide, which is capable of acting as a point of initiation of synthesis when placed under conditions in which synthesis of primer extension product which is complementary to a nucleic acid strand (template) is induced, i.e., in the presence of nucleotides and an agent for polymerization, such as DNA polymerase, and at a suitable temperature and pH. In particular, the primer is a single-stranded deoxyribonucleotide molecule. The primer used in the present disclosure may comprise naturally occurring dNMPs (i.e., dAMP, dGMP, dCMP, and dTMP), modified nucleotides, or non-natural nucleotides. In addition, conventional primers may include ribonucleotides.

The primer must be sufficiently long to prime the synthesis of extension products in the presence of the agent for polymerization. The suitable length of the primer depends on many factors, including temperature, application, and source of primer. As used herein, the term “annealing” or “priming” means the apposition of an oligodeoxynucleotide or nucleic acid onto a template nucleic acid, which causes a polymerase to polymerize nucleotides to form a nucleic acid molecule complementary to the template nucleic acid or a portion thereof.

As used herein, the term “self-dimer primer” refers to a primer capable of partially hybridizing with another self-dimer primer under certain conditions to form a self-dimer.

As used herein, the term “self-dimer” refers to a dimer formed by partial hybridization, i.e., by inter-strand hybridization, between two self-dimer primers identical to each other.

Conventional primers serve to initiate extension after hybridization to a nucleic acid strand (template), whereas the self-dimer primer according to the present disclosure serves to initiate extension after hybridization to another self-dimer primer.

As used herein, another self-dimer primer that hybridizes to the self-dimer primer refers to a self-dimer primer having the same sequence as the self-dimer primer. That is, according to the method of the present disclosure, two self-dimer primers of the same sequence are hybridized to each other.

In a nucleic acid amplification reaction, for example, PCR, a primer dimer generally means that primers form a specific or non-specific bond between them (S. Das, S. C. Mohapartra, J. T. Hsu, Biotechnol. Tech. 1999, 13, 643-646). The primer dimer is amplified by an activated nucleic acid polymerase in a PCR reagent to cause loss of primers, which may lead to poor results such as inhibited production of final amplification products, reduced signals (e.g., reduced RFU values), or varied cycle threshold (Ct) values. Accordingly, attempts have been made to inhibit the formation of primer dimers by designing primers to have a sequence in which binding between primers can be minimized (P. M. Vallone, J. M. Butler, Biotechniques, 2018, 37(2), 226-231), chemically modifying primers (U.S. Pat. Nos. 6,183,998, 5,773,258, 5,677,152, 5,338,671, 6,403,341; Korean Patent No. 10-1424192), adding a hot-start enzyme or additional single-stranded DNA binding material that restores activity only at high temperatures (C. J. Kubu, Biotechniuqes 2018, 44(2), 275-277), or adding additives such as DMSO to disrupt binding between DNAs (C. M. Sedgley, A. C. Nagel, C. E. Shelburne, D. B. Clewell, O. Appelbe, A. Molander, Archives of Oral Biology, 2005, 50(6), 575-583).

Such attempts have been focused on reducing or removing the formation of primer dimers as much as possible, and there is no research on how to form primer dimers artificially and utilize them.

The self-dimer primer used in the method of the present disclosure comprises, in the 5′ to 3′ direction, (i-1) a 5′-hairpin-non-forming portion and (i-2) a 3′-hairpin-forming portion.

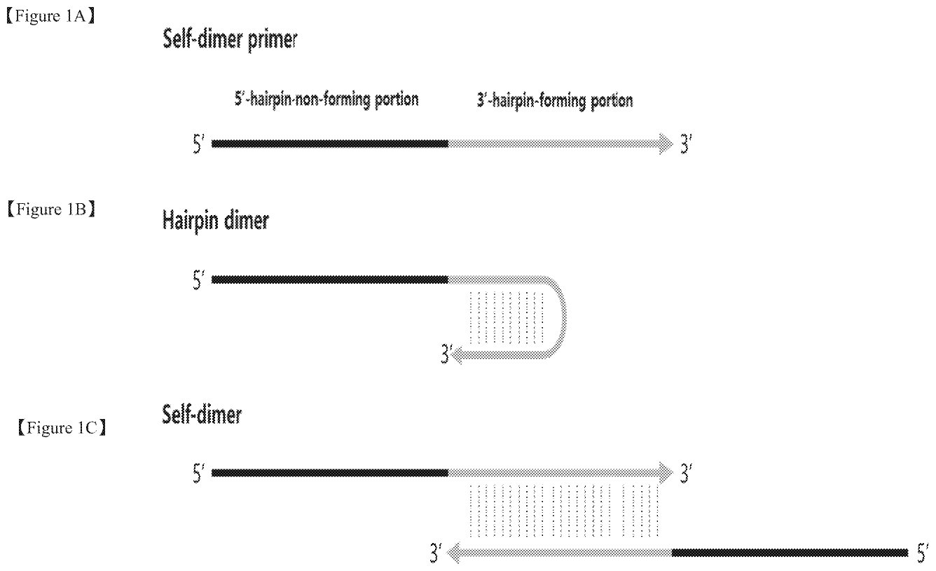

The structure of the self-dimer primer according to the present disclosure is illustrated in FIG. 1.

(i-1) 5′-Hairpin-Non-Forming Portion

As used herein, the term “5′-hairpin-non-forming portion” in connection with the self-dimer primer refers to a portion at the 5′ end of the self-dimer primer except for the 3′-hairpin-forming portion. The 5′-hairpin-non-forming portion is indicated by a solid black line in FIG. 1A.

Specifically, the term “5′-hairpin-non-forming portion” refers to a portion which is not involved in forming a hairpin (hairpin dimer) and serves as a template for extension of another self-dimer primer hybridized to the self-dimer primer.

The 5′-hairpin-non-forming portion of the self-dimer primer has a nucleotide sequence substantially non-complementary to the 5′-hairpin-non-forming portion and the 3′-hairpin-forming portion of another self-dimer primer. Thus, the 5′-hairpin-non-forming portion of the self-dimer primer does not substantially hybridize to the 5′-hairpin-non-forming portion or the 3′-hairpin-forming portion of another self-dimer primer.

The term “non-complementary” refers that a primer or probe is sufficiently non-complementary so as not to selectively hybridize to a target nucleic acid under predetermined annealing conditions or stringent conditions, encompassing “substantially non-complementary” and “perfectly non-complementary”. The term “non-complementary” in connection with the 5′-hairpin-non-forming portion of the self-dimer primer refers that the 5′-hairpin-non-forming portion of the self-dimer primer is sufficiently non-complementary so as not to be selectively hybridized to any portion of another self-dimer primer under predetermined annealing conditions or stringent conditions.

The 5′-hairpin-non-forming portion of the self-dimer primer according to the present disclosure does not have a self-complementary sequence. Therefore, the 5′-hairpin-non-forming portion does not form a hairpin (hairpin dimer).

The 5′-hairpin-non-forming portion of a self-dimer primer herein can be any sequence as long as it does not form a hairpin dimer and has a nucleotide sequence that is substantially non-complementary to another self-dimer primer.

In one embodiment, the 5′-hairpin-non-forming portion of the self-dimer primer is 5 to 50 nucleotides in length. For example, the 5′-hairpin-non-forming portion of the self-dimer primer is 7 to 50, 10 to 50, 15 to 50, 20 to 50, 25 to 50, 30 to 50, 35 to 50, 40 to 50, 45 to 50, 7 to 45, 10 to 45, 15 to 45, 20 to 45, 25 to 45, 30 to 45, 35 to 45, 40 to 45, 7 to 40, 10 to 40, 15 to 40, 20 to 40, 25 to 40, 30 to 40, 35 to 40, 7 to 35, 10 to 35, 15 to 35, 20 to 35, 25 to 35, 30 to 35, 7 to 30, 10 to 30, 15 to 30, 20 to 30, 25 to 30, 7 to 25, 10 to 5, 15 to 25, 20 to 25, 7 to 20, 10 to 20, 15 to 20, 7 to 5, 10 to 15, or 7 to 10 nucleotides in length.

The method of the present disclosure utilize the signal provided by extension of the self-dimer primer along the 5′-hairpin-non-forming portion of another self-dimer primer to measure the activity of a nucleic acid polymerase. If the 5′-hairpin-non-forming portion is too short, the signal generated by the extension of the self-dimer primer may be insignificant, thereby making it difficult to accurately measure the activity of the nucleic acid polymerase. In contrast, if the 5′-hairpin-non-forming portion is too long, the synthesis efficiency of the self-dimer primer may be low and a linear function of signal versus time at the early phase of incubation may not be achieved.

The self-dimer primer according to the present disclosure should not be blocked at the 3′ end for extension by a nucleic acid polymerase.

Prior to incubating, the self-dimer primer according to the present disclosure may be present in various forms as follows: (i) a single strand as illustrated in FIG. 1A, (ii) a hairpin dimer by intra-strand hybridization as illustrated in FIG. 1B, or (iii) a self-dimer by inter-strand hybridization as illustrated in FIG. 1C, depending on temperature conditions.

Thus, even if the self-dimer primer is present in any of the forms (i)-(iii), the 5′-hairpin-non-forming portion of the self-dimer primer is always single-stranded prior to incubating.

(i-2) 3′-Hairpin-Forming Portion

As used herein, the term “3′-hairpin-forming portion” in connection with the self-dimer primer refers to a portion at the 3′ end of the self-dimer primer. The term refers to a portion which can form a hairpin (hairpin dimer) at a first temperature and form a self-dimer at a second temperature. The 3′-hairpin-forming portion is depicted by a solid gray line in FIG. 1A.

In an embodiment, the 3′-hairpin-forming portion of the self-dimer primer forms a hairpin dimer at a first temperature.

As used herein, the term “hairpin” or “hairpin dimer” refers to a structure having a double-stranded portion formed via intra-hybridization due to a self-complementary sequence in the 3′-hairpin-forming portion at a first temperature. The term refers to a structure having a double-stranded portion formed by a single self-dimer primer. The structure of the hairpin dimer is illustrated in FIG. 1B.

As shown in FIG. 1B, at a first temperature, the 3′-hairpin-forming portion of the self-dimer primer of the present disclosure forms a hairpin dimer having a double-stranded portion via intra-strand hybridization.

The first temperature is predetermined based on the melting temperature (Tm) of the hairpin dimer. Specifically, the first temperature may be equal to or lower than the Tm of the hairpin dimer. At a temperature equal to or lower than the Tm of the hairpin dimer, the 3′-hairpin-forming portion of the self-dimer primer may form a hairpin dimer as illustrated in FIG. 1B or a self-dimer as illustrated in FIG. 1C.

Since the length of the double-stranded portion of the hairpin dimer is approximately half the length of the double-stranded portion of the self-dimer, the Tm of the hairpin dimer is lower than the Tm of the self-dimer. Therefore, at a temperature below the Tm of the hairpin dimer, both the self-dimer and the hairpin dimer may be formed.

In an embodiment, the 3′-hairpin-forming portion of the self-dimer primer hybridizes with the 3′-hairpin-forming portion of another self-dimer primer to form a self-dimer at a second temperature.

As used herein, the term “self-dimer” refers to a structure having a double-stranded portion formed by hybridization between the 3′-hairpin-forming portions, i.e., inter-hybridization, of two self-dimers at a second temperature. Both strands of the self-dimer may be extended in the direction of 5′ to 3′ at the working temperature of the nucleic acid polymerase.

The structure of the self-dimer is illustrated in FIG. 1C.

As illustrated in FIG. 1C, at a second temperature, the 3′-hairpin-forming portions of two self-dimer primers according to the present disclosure forms a self-dimer having a double-stranded portion by inter-strand hybridization.

In an embodiment, the second temperature is predetermined based on the Tm of the self-dimer. Specifically, the second temperature may be equal to or lower than the Tm of the self-dimer. At a temperature equal to or lower than the Tm of the self-dimer, the 3′-hairpin-forming portion of the self-dimer primer may form a self-dimer as in FIG. 1C.

In certain embodiments, the second temperature is predetermined based on the Tm of the self-dimer and the Tm of the hairpin dimer. Specifically, the second temperature may be higher than the Tm of the hairpin dimer and equal to or lower than the Tm of the self-dimer. At a temperature higher than the Tm of the hairpin dimer, the 3′-hairpin-forming portion of the self-dimer primer does not form a hairpin dimer as in FIG. 1B, while at a temperature equal to or lower than the Tm of the self-dimer, the 3′-hairpin-forming portions of two self-dimer primers can form a self-dimer as in FIG. 1C.

In certain embodiments, the second temperature is higher than the first temperature.

In order to form a hairpin dimer or self-dimer as described above, the 3′-hairpin-forming portion of the self-dimer primer may have a nucleotide sequence hybridizable to the 3′-hairpin-forming portion of another self-dimer primer.

As used herein, the term “hybridization” or “hybridizing” means that complementary single-stranded nucleic acids form a double-stranded nucleic acid. Hybridization may occur between two nucleic acid strands that are either perfectly complementary or substantially complementary despite some non-complementarity. Complementarity for hybridization may vary depending on the hybridization conditions, particularly temperature.

Hybridization between two self-dimer primers may be carried out under suitable hybridization conditions generally determined by the optimization procedure. Conditions such as temperature, concentration of the components, number of hybridization and washes, and composition, pH, and ionic strength of buffer may vary depending on various factors including the length and GC content of the self-dimer primer. For example, when using a self-dimer primer pair having a relatively short 3′-hairpin-forming portion, it is desirable to select a low stringent condition. Detailed conditions for hybridization can be found in Joseph Sambrook, et al., Molecular Cloning, A Laboratory Manual, Cold Spring Harbor Laboratory Press, Cold Spring Harbor, N.Y. (2001); and M. L. M. Anderson, Nucleic Acid Hybridization, Springer-Verlag New York Inc. N.Y. (1999).

The terms “annealing” and “hybridization” are used interchangeably.

According to the present disclosure, the 3′-hairpin-forming portion of the self-dimer primer has a nucleotide sequence substantially complementary to the 3′-hairpin-forming portion of another self-dimer primer.

The term “complementary” or “complementarity” in the context of the 3′-hairpin-forming portion of the self-dimer primer means that the 3′-hairpin-forming portion of the self-dimer primer is sufficiently complementary to selectively hybridize to the 3′-hairpin-forming portion of another self-dimer primer under certain annealing conditions or stringent conditions. The term encompasses “substantially complementary” and “perfectly complementary,” preferably being perfectly complementary.

In particular, the 3′ end of the self-dimer primer according to the present disclosure is necessarily complementary to another self-dimer primer so as to be extended by polymerase. Specifically, one or more nucleotides, such as one, two, or three nucleotides, at the 3′ end of the self-dimer primer according to the present disclosure are complementary to nucleotides at the corresponding position of another self-dimer primer.

In one embodiment, the 3′-hairpin-forming portion of the self-dimer primer according to the present disclosure has a substantially palindromic sequence.

As used herein, the term “palindromic sequence” means that the k^{th} nucleotide base from the 5′-terminus is complementary to the k^{th} nucleotide base from the 3′-terminus, wherein k is an integer of 1 or greater.

In one embodiment, the 3′-hairpin-forming portion of the self-dimer primer according to the present disclosure consists of a palindromic sequence.

In certain embodiments, when the 3′-hairpin-forming portion of the self-dimer primer is n nucleotides in length, the m^{th} nucleotide base from the 3′-terminus is complementary to the n−m+1^{th} nucleotide base, wherein n is an integer from 5 to 50, and m is an integer of 1 or greater.

For example, when the 3′-hairpin-forming portion is 10 nucleotides in length (n=10), which has the nucleotide sequence as follows:
5′-a-b-c-d-e-f-g-h-i-j-3′
wherein the first (m=1) nucleotide base “j” from the 3′-terminus should be complementary to the 10^{th} (n−m+1=10) nucleotide base “a”, the second (m=2) nucleotide base “i” from the 3′-terminus should be complementary to the 9^{th} (n−m+1=9) nucleotide base “b”, and the same applies to other nucleotide bases.

The 3′-hairpin-forming portion of the self-dimer primer according to the present disclosure may comprise a nucleotide non-complementary to the 3′-hairpin-forming portion of another self-dimer primer.

According to an embodiment, the 3′-hairpin-forming portion of the self-dimer primer has one or more non-complementary nucleotides, such as 1, 2, 3, 4, 5, or 6 non-complementary nucleotides, so long as it does not significantly affect the formation of self-dimers by hybridization between the 3′-hairpin-forming portions and the extension of both strands of the self-dimers. The 3′-hairpin-forming portions of two self-dimer primers can be successfully hybridized and extended depending on the incubation conditions, even if there are some non-complementary nucleotides therein.

The non-complementary nucleotides may be exemplified as follows.

As an example, when the 3′-hairpin-forming portion is 10 nucleotides in length (n=10), which has the nucleotide sequence as follows:
5′-a-b-c-d-e-f-g-h-i-j-3′
wherein the 2^{nd} nucleotide base “i” from the 3′-terminus is non-complementary to the 9^{th} nucleotide base “b”, while the other nucleotide bases may be complementary.

As another example, when the 3′-hairpin-forming portion is 11 nucleotides in length (n=11), which has the nucleotide sequence as follows:
5′-a-b-c-d-e-f-g-h-i-j-k-3′
wherein the 6^{th} nucleotide base “f” from the 3′-terminus is non-complementary, while the other nucleotide bases may be complementary.

The non-complementary nucleotides may be consecutive or nonconsecutive in the 3′-hairpin-forming portion.

According to an embodiment of the present disclosure, the 3′-hairpin-forming portion of the self-dimer primer has at least 2, at least 3, at least 4, at least 5, at least 6, at least 7, or at least 8 nonconsecutive complementary nucleotides, including a nucleotide at the 3′-end.

According to an embodiment of the present disclosure, the 3′-hairpin-forming portion of the self-dimer primer has a nucleotide sequence perfectly complementary to the 3′-hairpin-forming portion of another self-dimer primer.

The 3′-hairpin-forming portion of the self-dimer primer herein can be any sequence as long as it forms a hairpin dimer at a first temperature and forms a self-dimer at a second temperature.

In certain embodiments, the 3′-hairpin-forming portion of the self-dimer primer is between 5 and 50 nucleotides in length. For example, the 3′-hairpin-forming portion of the self-dimer primer is 7 to 50, 10 to 50, 15 to 50, 20 to 50, 25 to 50, 30 to 50, 35 to 50, 40 to 50, 45 to 50, 7 to 45, 10 to 45, 15 to 45, 20 to 45, 25 to 45, 30 to 45, 35 to 45, 40 to 45, 7 to 40, 10 to 40, 15 to 40, 20 to 40, 25 to 40, 30 to 40, 35 to 40, 7 to 35, 10 to 35, 15 to 35, 20 to 35, 25 to 35, 30 to 35, 7 to 30, 10 to 30, 15 to 30, 20 to 30, 25 to 30, 7 to 25, 10 to 5, 15 to 25, 20 to 25, 7 to 20, 10 to 20, 15 to 20, 7 to 5, 10 to 15, or 7 to 10 nucleotides in length.

In certain embodiments, when the 3′-hairpin-forming portion of the self-dimer primer hybridizes to the 3′-hairpin-forming portion of another self-dimer primer to form a self-dimer, the Tm of the self-dimer is higher than the incubation temperature according to the method of the present disclosure. Adjusting the Tm of the self-dimer and the incubation temperature allows the formation of the self-dimer during incubation.

In certain embodiments, when the 3′-hairpin-forming portion of the self-dimer primer hybridizes with the 3′-hairpin-forming portion of another self-dimer primer to form a self-dimeric, the Tm of the self-dimer is higher than the optimum temperature for activity of the nucleic acid polymerase. Adjusting the Tm of the self-dimer and the optimum temperature allows the self-dimer to be extended at the optimum temperature during incubation.

In an embodiment, the ratio of the length of the 5′-hairpin-non-forming portion to the length of the 3′-hairpin-forming portion is 1:5 to 5:1.

In certain embodiments, the ratio of the length of the 5′-hairpin-non-forming portion to the length of the 3′-hairpin-forming portion is about 1:1.

In certain embodiments, the ratio of the length of the 5′-hairpin-non-forming portion to the length of the 3′-hairpin-forming portion is about 1:2.

In certain embodiments, the ratio of the length of the 5′-hairpin-non-forming portion to the length of the 3′-hairpin-forming portion is about 1:3.

In certain embodiments, the ratio of the length of the 5′-hairpin-non-forming portion to the length of the 3′-hairpin-forming portion is about 1:4.

An appropriate ratio of the lengths of the two portions can improve the provision of signals upon extension of the self-dimer (upon formation of the extended dimer) and the linear correlation of the function of signal versus time, which leads to increased sensitivity and accuracy in the analysis of activity of the nucleic acid polymerase.

In one embodiment, the self-dimer primer according to the present disclosure is 10 to 100 nucleotides in length. For example, the self-dimer primers may be 10 to 100, 10 to 95, 10 to 90, 10 to 85, 10 to 80, 10 to 75, 10 to 70, 10 to 65, 10 to 60, 10 to 55, 10 to 50, 10 to 45, 10 to 40, 10 to 35, 10 to 30, 10 to 25, 10 to 20, 10 to 15, 15 to 100, 15 to 95, 15 to 90, 15 to 85, 15 to 80, 15 to 75, 15 to 70, 15 to 65, 15 to 60, 15 to 55, 15 to 50, 15 to 45, 15 to 40, 15 to 35, 15 to 30, 15 to 25, 15 to 20, 20 to 100, 20 to 95, 20 to 90, 20 to 85, 20 to 80, 20 to 75, 20 to 70, 20 to 65, 20 to 60, 20 to 55, 20 to 50, 20 to 45, 20 to 40, 20 to 35, 20 to 30, 20 to 25, 25 to 100, 25 to 95, 25 to 90, 25 to 85, 25 to 80, 25 to 75, 25 to 70, 25 to 65, 25 to 60, 5 to 55, 25 to 50, 25 to 45, 25 to 40, 25 to 35, 25 to 30, 30 to 100, 30 to 95, 30 to 90, 30 to 85, 30 to 80, 30 to 75, 30 to 70, 30 to 65, 30 to 60, 30 to 55, 30 to 50, 30 to 45, 30 to 40, 30 to 35, 35 to 100, 35 to 95, 35 to 90, 35 to 85, 35 to 80, 35 to 75, 35 to 70, 35 to 65, 35 to 60, 35 to 55, 35 to 50, 35 to 45, 35 to 40, 40 to 100, 40 to 95, 40 to 90, 40 to 85, 40 to 80, 40 to 75, 40 to 70, 40 to 65, 40 to 60, 40 to 55, 40 to 50, 40 to 45, 45 to 100, 45 to 95, 45 to 90, 45 to 85, 45 to 80, 45 to 75, 45 to 70, 45 to 65, 45 to 60, 45 to 55, 45 to 50, 50 to 100, 50 to 95, 50 to 90, 50 to 85, 50 to 80, 50 to 75, 50 to 70, 50 to 65, 50 to 60, 50 to 55, 55 to 100, 55 to 95, 55 to 90, 55 to 85, 55 to 80, 55 to 75, 55 to 70, 55 to 65, 55 to 60, 60 to 100, 60 to 95, 60 to 90, 60 to 85, 60 to 80, 60 to 75, 60 to 70, 60 to 65, 65 to 100, 65 to 95, 65 to 90, 65 to 85, 65 to 80, 65 to 75, 65 to 70, 70 to 100, 70 to 95, 70 to 90, 70 to 85, 70 to 80, 70 to 75, 75 to 100, 75 to 95, 75 to 90, 75 to 85, 75 to 80, 80 to 100, 80 to 95, 80 to 90, 80 to 85, 85 to 100, 85 to 95, 85 to 90, 90 to 100, 90 to 95, or 95 to 100 nucleotides in length.

It will be understood that the length of the self-dimer primer may be adjusted by those skilled in the art based on several factors including sensitivity of the analysis, incubation temperature, and synthesis efficiency of self-dimer primers.

Unlike conventional primers, the self-dimer primer according to the present disclosure may serve as a primer, a probe, and a template.

First, the self-dimer primer of the present disclosure can serve as a primer which hybridizes to another self-dimer primer and then is extended. Second, the self-dimer primers of the present disclosure can serve as a probe which provides a detectable signal from an intercalating dye upon formation of an extended dimer. Third, the self-dimer primer of the present disclosure can serve as a template for hybridization with and extension of another self-dimer primer. Thus, the method of the present disclosure do not require any additional template, primer, and probe. In particular, the method of the present disclosure is performed in the absence of a template specific for the self-dimer primer, i.e., a template that hybridizes with the self-dimer primer.

The self-dimer primer may be composed of naturally occurring dNMPs. Alternatively, the self-dimer primer may comprise modified or non-natural nucleotides, such as PNA (Peptide Nucleic Acid; see WO 92/20702) and LNA (Locked Nucleic Acid; see WO 98/22489, WO 98/39352, and WO 99/14226). In addition, the self-dimer primer may include universal bases such as deoxyinosine, inosine, 1-(2′-deoxy-beta-D-ribofuranosyl)-3-nitropyrrole, and 5-nitroindole. The term “universal base” means a base capable of base pairing with each of natural DNA/RNA bases with no discrimination.

The modified nucleotides, the non-natural nucleotides, or the universal bases may consecutively or nonconsecutively exist in the self-dimer primer. The number of modified nucleotides, non-natural nucleotides, or universal bases may be 1-5, 1-3, or 1-2. The modified nucleotides, the non-natural nucleotides, or the universal bases may be present at any position in the self-dimer primer. In an embodiment of the present disclosure, the modified nucleotides, non-natural nucleotides, or universal bases may be located in the 5′-hairpin-non-forming portion, the 3′-hairpin-forming portion, or both.

The self-dimer primer of the present disclosure may comprise any additional sequence in addition to the 5′-hairpin-non-forming portion and the 3′-hairpin-forming portion. According to an embodiment of the present disclosure, the additional sequence is located toward the 5′ direction of the 5′-hairpin-non-forming portion, does not serve as a template, and does not hybridize with another self-dimer primer. The additional sequence may comprise a block to prohibit extension of the primer.

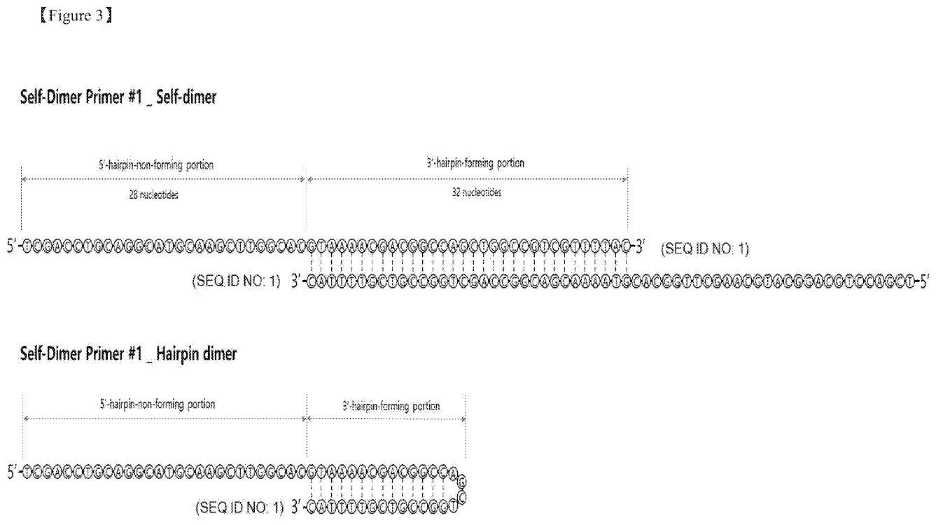

The structures of self-dimer primers used in this example are shown in FIGS. 3, 6, 7, and 8.

As illustrated in FIG. 3, Self-Dimer Primer #1 can form a self-dimer (top) or a hairpin dimer (bottom). Self-Dimer Primer #1 consists of a 5′-hairpin-non-forming portion of 28 nucleotides in length and a 3′-hairpin-forming portion of 32 nucleotides in length.

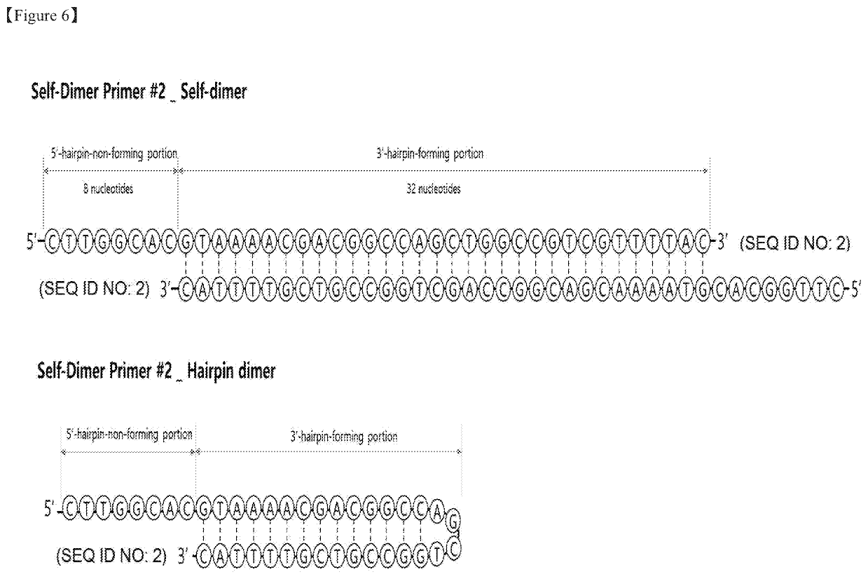

As illustrated in FIG. 6, Self-Dimer Primer #2 can form a self-dimer (top) or a hairpin dimer (bottom). Self-Dimer Primer #2 consists of a 5′-hairpin-non-forming portion of 8 nucleotides in length and a 3′-hairpin-forming portion of 32 nucleotides in length.

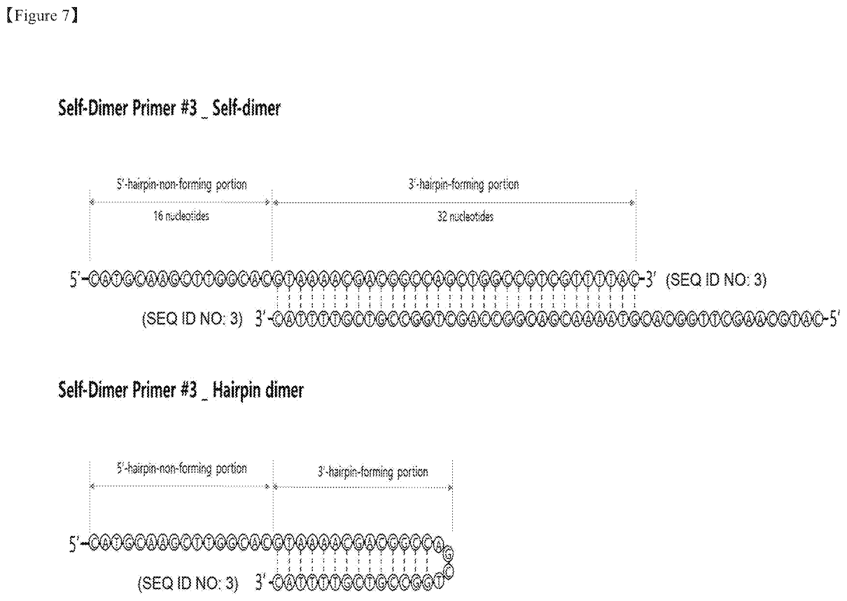

As illustrated in FIG. 7, Self-Dimer Primer #3 can form a self-dimer (top) or a hairpin dimer (bottom). Self-Dimer Primer #3 consists of a 5′-hairpin-non-forming portion of 16 nucleotides in length and a 3′-hairpin-forming portion of 32 nucleotides in length.

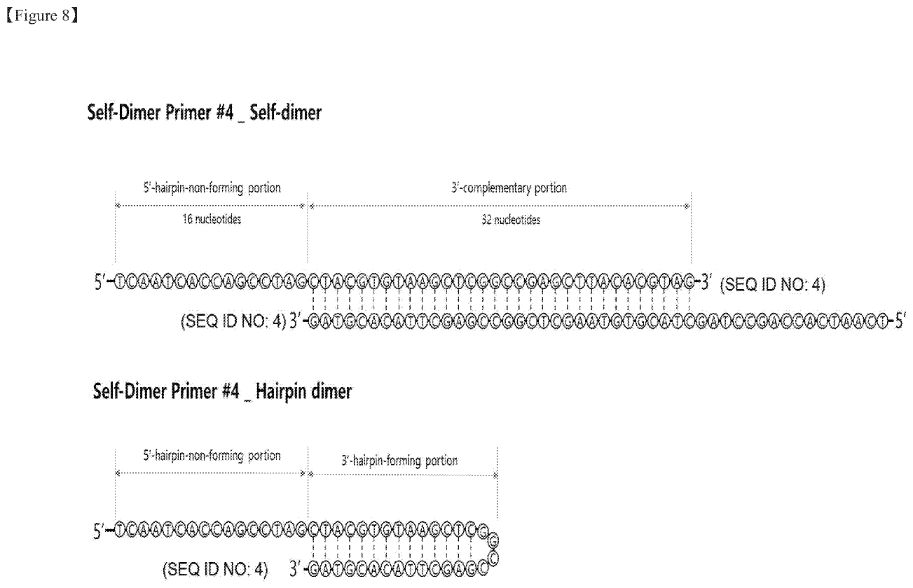

As illustrated in FIG. 8, Self-Dimer Primer #4 can form a self-dimer (top) or a hairpin dimer (bottom). Self-Dimer Primer #4 consists of a 5′-hairpin-non-forming portion of 16 nucleotides in length and a 3′-hairpin-forming portion of 32 nucleotides in length.

As described above, selection of an appropriate incubation temperature makes the formation of self-dimers superior to the formation of hairpin dimers.

### (ii) Intercalating Dye

The composition for analyzing activity according to the present disclosure comprises an intercalating dye which provides a detectable signal indicative of the presence of an extended dimer.

Examples of intercalating dyes useful in the present disclosure include, but are not limited to, EvaGreen, ethidium bromide, SYBR Green I, SYBR Gold, Yo-Yo, Yo-Pro, TOTO, SYTO9, BEBO, BOXTO, Hoechst dyes, LC Green Plus, ResoLight, Chromofy, and PicoGreen.

Intercalating dyes are intercalated into double-stranded nucleic acid molecules to generate signals. Prior to incubation, a relatively small number of intercalating dyes are intercalated into the hairpin dimers (having a relatively short double-stranded portion), whereas during incubation, a relatively large number of intercalating dyes are intercalated into the extended dimers, resulting in a relatively high signal intensity. This signal intensity increases as the number of extended dimers increases during incubation.

The signal provided by the intercalating dye described above is a fluorescence signal, and an increase in the fluorescence signal is expressed as an increase in the intensity of the fluorescence signal.

(iii) dNTPs or NTPs

The composition for analyzing activity according to the present disclosure may comprise dNTPs or NTPs as building blocks of DNA or RNA to be synthesized. The dNTPs comprise a mixture of four deoxyribonucleotides, i.e., dATP, dCTP, dGTP, and dTTP (or dUTP). The NTPs comprise a mixture of four ribonucleotides, i.e., ATP, CTP, GTP, and UTP.

The composition for analyzing activity according to the present disclosure may further comprise a buffer, a salt, or an additive to aid in the extension of both strands of the self-dimer by the nucleic acid polymerase.

The buffer as described above may serve to provide a pH environment for optimal activity of the nucleic acid polymerase and to prevent rapid changes in pH due to temperature changes and chemical interaction during the extension reaction by the nucleic acid polymerase. An example of the buffer includes Tris-HCl.

The salt as described above may serve to stabilize the activity of the nucleic acid polymerase. The salt may be a salt of magnesium or potassium, for example, MgCl₂, MgSO₄, or KCl. The salt may be added at various concentrations known in the art.

The additives as described above may serve to stabilize the nucleic acid polymerase. Examples of such additives include BSA, DMSO, betaine, KCl, and non-ionic surfactants (e.g., Tween 20, Triton X-100).

In addition to the foregoing, it is understood that various buffers, salts, or additives to be used in conventional nucleic acid amplification reactions may be used.

In addition, it is to be understood that the composition for analyzing activity according to the present disclosure may further comprise any other components known in the art.

However, the composition for analyzing activity according to the present disclosure does not include an additional oligonucleotide other than the self-dimer primer.

### Incubation (or Incubating)

The method of the present disclosure comprise incubating the nucleic acid polymerase of interest with the composition for analyzing activity as described above.

As used herein, the term “incubation” or “incubating” means that the nucleic acid polymerase of interest and the composition for analyzing activity are placed under reaction conditions allowing the formation of a self-dimer from the self-dimer primers and then the formation of an extended dimer by the activity of the nucleic acid polymerase.

The term “incubation” or “incubating” may be used interchangeably with “reaction” or “polymerization reaction”.

As described above, prior to incubation, there may be (i) the single-stranded self-dimer primer as illustrated in FIG. 1A, (ii) a hairpin dimer by intra-strand hybridization of the self-dimer as illustrated in FIG. 1B, or (iii) a self-dimer by inter-strand hybridization of two self-dimer primers as illustrated in FIG. 1C, depending on the temperature.

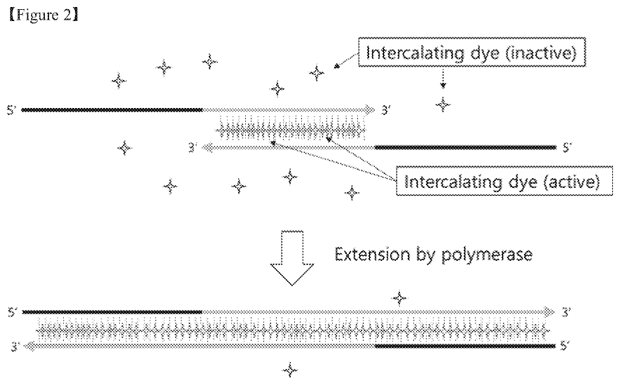

Then, during incubation, the 3′-hairpin-forming portions of the two self-dimer primers hybridize to each other to form a self-dimer, both strands of the resulting self-dimer are extended by the activity of the nucleic acid polymerase of interest to form an extended dimer, and the formation of the extended dimer provides a detectable signal from an intercalating dye (see FIG. 2).

The incubation in the method according to the present disclosure should prevent the self-dimer primer from being present as a single strand as shown in FIG. 1A, and prevent two self-dimer primers from forming a hairpin dimer by intra-strand hybridization as shown in FIG. 1B.

In one embodiment, the incubation is performed under isothermal conditions such that the nucleic acid polymerase of interest functions and the self-dimer primer is not present as a single strand.

In one embodiment, the incubation is performed under isothermal conditions such that the nucleic acid polymerase of interest functions and two self-dimer primers do not form a hairpin dimer by intra-strand hybridization.

In one embodiment, the incubation is performed under isothermal conditions such that the nucleic acid polymerase of interest functions and the self-dimer primer is not present as a single strand and does not form a hairpin dimer by intra-strand hybridization.

In one embodiment, the incubation is performed under isothermal conditions such that the 3′-hairpin-forming portions of the two self-dimer primers hybridize to each other to form a self-dimer.

In one embodiment, the incubation is performed under isothermal conditions such that both strands of the self-dimer are extended by the activity of the nucleic acid polymerase of interest to form an extended dimer.

In certain embodiments, the incubation is performed under an isothermal condition such that the self-dimer primer does not form a hairpin dimer, but two self-dimer primers form a self-dimer by inter-strand hybridization, followed by forming an extended dimer.

The function of the nucleic acid polymerase of interest, the non-formation of the hairpin dimer, the formation of the self-dimer, and the formation of the extended dimer as described above can be achieved by adjusting the temperature for the incubation.

The incubation temperature to achieve the above objects may be selected based on the temperature for the function of the nucleic acid polymerase, the Tm of the hairpin dimer, the Tm of the self-dimer, and the Tm of the extended dimer.

The incubation temperature may be the optimal temperature for the activity of the nucleic acid polymerase of interest. Therefore, the incubation temperature may vary depending on the optimum temperature for the activity of the nucleic acid polymerase of interest.

In one embodiment, the incubation temperature is higher than the Tm of the hairpin dimer for non-formation of the hairpin dimer.

In one embodiment, the incubation temperature is lower than the Tm of the self-dimer for formation of the self-dimer.

In one embodiment, the incubation temperature is lower than the Tm of the extended dimer for formation of the extended dimer.

In one embodiment, the incubation temperature is a temperature selected from 65 to 80° C. In certain embodiments, the incubation temperature is about 65° C. In certain embodiments, the incubation temperature is about 72° C.

According to the present disclosure, the hairpin dimer formed by intra-strand hybridization of the self-dimer primer and a self-dimer formed by inter-strand hybridization of two self-dimer primers have double-stranded portions of different lengths. The length of the double-stranded portion of the self-dimer is approximately twice the length of the double-stranded portion of the hairpin dimer. Thus, according to the method of the present disclosure, the Tm of the double-stranded portion of the self-dimer is higher than the Tm of the double-stranded portion of the unwanted hairpin dimer. This difference in Tm of these two dimers allows only self-dimers to be formed by adjusting the incubation temperature.

The method of the present disclosure may further comprise heating the mixture of the nucleic acid polymerase of interest and the composition for analyzing activity to a high temperature, prior to said incubation. The heating may render the self-dimer primer according to the present invention single-stranded as illustrated in FIG. 1A. Then, the heated mixture may be cooled to an incubation temperature or a temperature lower than that to induce the formation of the self-dimer. The high temperature is a temperature at which the self-dimer primer according to the present disclosure may be single stranded as described above. The high temperature is higher than an incubation temperature. In one embodiment, the high temperature is selected from 90° C., 91° C., 92° C., 93° C., 94° C., 95° C., 96° C., 97° C., 98° C., 99° C., 100° C., and any value therebetween. In certain embodiments, the high temperature is about 100° C.

According to one embodiment, the incubation is carried out for 30 minutes to 60 minutes. However, the incubation time may be adjusted in consideration of the time required to reach a plateau in the plot of signal versus time, or the time required for a linear function to appear in the plot of signal versus time. For example, if the time required to reach a plateau in the plot of signal versus time for a particular nucleic acid polymerase is greater than 60 minutes, the incubation time may be set to greater than 60 minutes for the same or similar type of nucleic acid polymerases. Likewise, if the time required to reach a plateau in the plot of signal versus time for a particular nucleic acid polymerase is within 30 minutes, the incubation time may be set to within 30 minutes for the same or similar type of nucleic acid polymerases.

In one embodiment, the incubation is carried out in an incubator well known in the art.

In another embodiment, the incubation is carried out in an amplification device used for conventional nucleic acid amplification reactions. The amplification device includes a thermal cycler capable of controlling a temperature and a detector capable of detecting a signal. Typically, amplification devices are programmed to continuously vary the temperature, e.g., in order to perform PCR, but can be used in the method of the present disclosure by being programmed to remain isothermal.

Examples of amplification devices that may be used include, but are not limited to, CFX (Bio-Rad), iCycler (Bio-Rad), LightCycler (Roche), StepOne (ABI), 7500 (ABI), ViiA7 (ABI), QuantStudio (ABI), and AriaMx (Agilent).

In the method of the present disclosure, the detectable signal is provided dependent on the presence of the extended dimer.

As used herein, the term “signal” refers to any signal, particularly a fluorescence signal, that can indicate the presence or absence of the extended dimer. For example, the signal includes a signal (signal generation or extinction) or change in the signal (signal increase or decrease) from the intercalating dye.

### Step (b): Measuring a Detectable Signal

Next, the detectable signal is measured over time during incubation. The change in signal over time is indicative of the activity of the nucleic acid polymerase of interest.

The measurement of the detectable signals herein is carried out in real time or at predetermined time intervals at one temperature, i.e., isothermal.

When a nucleic acid polymerase is analyzed by the method according to the present disclosure, the signal intensity (fluorescence value) increases constantly, i.e., a constant rate of change in signal is observed, in the early phase of incubation. However, when the incubation time further elapses, the rate of change of the signal gradually decreases to zero (0).

That is, when a nucleic acid polymerase is analyzed for activity according to the method of the present disclosure, a plot of signal intensity versus incubation time in which signal intensity increases and then no longer increases over time, i.e., a plot of rate of change in signal versus incubation time in which the rate of change in signal has a constant value exceeding zero and then gradually decreases to zero over time is obtained.

This is because, in the early phase of incubation, the nucleic acid polymerase consumes the dNTP added for incubation and extends both strands of the self-dimer, resulting in a continuous increase in signal intensity, that is, a constant rate of change in signal, but when time elapses, the dNTPs is gradually depleted, resulting in a reduced rate of change in signal.

The constant rate of change in signal in the early phase of incubation may vary depending on the activity of the nucleic acid polymerase of interest. For example, the use of a nucleic acid polymerase having high activity results in a high rate of change in signal in the early phase of incubation, compared with the use of a nucleic acid polymerase having low activity at the same concentration. Therefore, a level or degree of a constant rate of change in signal in the early phase of incubation can indicate the activity of the nucleic acid polymerase.

In one embodiment, the results for the measurement of the detectable signal in step (b) are presented as a plot of signal intensity (fluorescence value) versus incubation time.

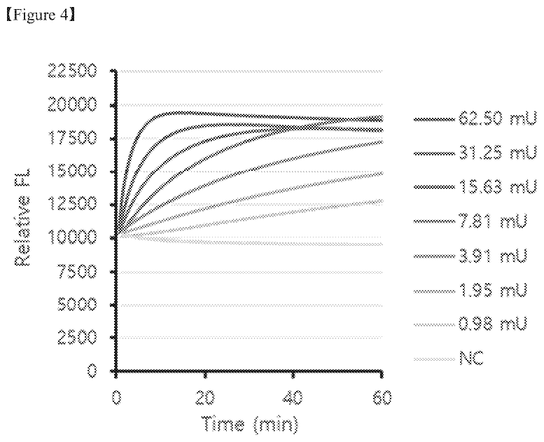

Exemplary plots are shown in FIGS. 4, 9, and 10. As shown in each drawing, the signal intensity continuously increases in an early phase of the incubation, but the increase in the signal intensity reaches a plateau in a late phase of the incubation.

In one embodiment, the change in signal is measured in an early phase of incubation.

As described above, the plot of signal intensity versus incubation time represents a linear function, i.e., a first-order function, in the early phase of incubation.

Thus, the change in signal is measured over a period of time during which a plot of signal intensity versus incubation time represents a linear function.

In one embodiment, the change in signal over time is determined by calculating a slope or first derivative from the linear function of signal intensity versus incubation time in the early phase of incubation.

The change in signal over time obtained by the method according to the present invention may be converted into another indicator which indicates the activity of a nucleic acid polymerase of interest.

In one embodiment, the change in signal overtime is converted into an amount of dNTP consumed in a unit time of incubation.

When the total amount of dNTP added to the incubation is known, the maximum signal value provided upon depletion of dNTPs is proportional to the total amount of dNTP added to the incubation, so that a signal value can be converted into an amount of dNTP consumed in a unit time of incubation.

The amount of dNTP consumed in a unit time of incubation may indicate the activity of the nucleic acid polymerase of interest. For example, when two different nucleic acid polymerases at the same concentration are analyzed for activity by the method of the present disclosure, it can be said that a nucleic acid polymerase having a large amount of dNTP consumed in a unit time of incubation has higher activity than a nucleic acid polymerase having a small amount of dNTP consumed in a unit time of incubation.

In this case, the activity of the nucleic acid polymerase refers to an amount of dNTP consumed in a unit time at a given temperature of incubation per unit concentration (mass) of the nucleic acid polymerase.

The activity of the nucleic acid polymerase of interest may be determined by the following steps: (i) determining the concentration of the nucleic acid polymerase, (ii) calculating the amount of dNTP consumed in a unit time, such as 10 minutes, 20 minutes, 30 minutes, 40 minutes, or any time therebetween, at said concentration, and (iii) dividing the amount of dNTP in step (ii) by the concentration of the nucleic acid polymerase in step (i) to calculate the amount of dNTP consumed in a unit time per unit concentration of the nucleic acid polymerase.

In step (i), the concentration of the nucleic acid polymerase of interest may be determined by one of various methods known in the art. As an example, the concentration of the nucleic acid polymerase of interest may be determined by the Bradford method, the BCA method, or the A280 absorbance. Alternatively, the concentration of the nucleic acid polymerase of interest may be determined by calculating the consumption of dNTP by a unit measurement of the nucleic acid polymerase of interest using radioisotopes. For example, when the concentration of the nucleic acid polymerase of interest representing the consumption of dNTP of 15 nmol for 30 minutes at 75° C. is defined as 1 unit by a unit measurement method using radioisotopes, the concentration of the nucleic acid polymerase of interest may be determined from the consumption of dNTP.

In step (ii), the amount of dNTP consumed in a unit time can be determined by converting the change in signal over time as described in the method (b) of the present disclosure into the amount of dNTP consumed in a unit time of incubation. The unit time may be appropriately selected by those skilled in the art, but is preferably 30 minutes adopted by a unit measurement using radioisotopes.

In step (iii), the amount of dNTP consumed in a unit time per unit concentration of the nucleic acid polymerase of interest is calculated by dividing the amount of dNTP in step (ii) by the concentration of the nucleic acid polymerase in step (i). For example, when the amount of dNTP consumed in 30 minutes in step (ii) is 249.82 pmol and the concentration of the nucleic acid polymerase of interest in step (i) is 62.50 mU, the amount of dNTP consumed in 30 minutes per unit concentration of the nucleic acid polymerase of interest is 3.997 pmol/mU (nmol/U).

The activity of a nucleic acid polymerase of interest as described above can be determined using a plurality of polymerases at different concentrations to improve its accuracy.

For example, the concentrations of a plurality of polymerases are determined, the amount of dNTP consumed in a unit time at each concentration is calculated, and a plot of the concentration of the polymerase versus the amount of dNTP consumed is obtained. In this case, the slope of the plot corresponds to the amount of dNTP consumed in a unit time per unit concentration of the polymerase.

As such, the method of the present disclosure enables determination of the activity of a polymerase of interest by converting the change in signal over time into an amount of dNTP consumed in a unit time per unit concentration of polymerase.

Since the method of the present disclosure uses a single type of self-dimer primer, it requires fewer factors to be considered in design and thus are easy to design, compared to conventional methods using both primers and templates. In addition, the conventional methods described above have disadvantages of inconsistent length of the final product, low signal consistency, and inaccurate analysis result due to the formation of unwanted dimers, whereas the method of the present disclosure can overcome these problems.

Further, the method of the present disclosure is simple, fast, cost-effective, safe, and user-friendly compared to conventional methods using radioisotopes.

Moreover, the method of the present disclosure can be applied to analyze the activity and purity and monitor quality control for various nucleic acid polymerases in their production. Further, it can be used in various places such as large hospitals, clinical laboratories, and research institutes to perform diagnosis of diseases using nucleic acid polymerases.

### II. Composition for Analyzing the Activity of a Nucleic Acid Polymerase of Interest

According to another aspect of the present disclosure, there is provided a composition for analyzing the activity of a nucleic acid polymerase of interest, comprising a self-dimer primer, wherein the self-dimer primer comprises, in the 5′ to 3′ direction, a 5′-hairpin-non-forming portion and a 3′-hairpin-forming portion, wherein during said incubating, the self-dimer primer hybridizes with another self-dimer primer between 3′-hairpin-forming portions to form a self-dimer, and both strands of the resulting self-dimer are extended by the activity of the nucleic acid polymerase of interest to form an extended dimer, wherein the formation of the extended dimer provides a detectable signal from the intercalating dye, wherein the change in signal over time is indicative of the activity of the nucleic acid polymerase.

Since the composition of the present disclosure is prepared to perform the method of aspect I according to the present disclosure, the common descriptions between them are omitted in order to avoid undue redundancy leading to the complexity of this specification.

According to an embodiment of the present disclosure, the composition further comprises an intercalating dye.

According to an embodiment of the present disclosure, the composition further comprises dNTPs or NTPs.

### III. Self-Dimer Primer

According to another aspect of the present disclosure, there is provided a self-dimer primer comprising, in the 5′ to 3′ direction, a 5′-hairpin-non-forming portion and a 3′-hairpin-forming portion, wherein the 5′-hairpin-non-forming portion has a nucleotide sequence substantially non-complementary to the 5′-hairpin-non-forming portion and the 3′-hairpin-forming portion of another self-dimer primer, and the 3′-hairpin-forming portion has a nucleotide sequence substantially complementary to the 3′-hairpin-forming portion of another self-dimer primer, wherein the self-dimer primer is capable of hybridizing with another self-dimer primer between 3′-hairpin-forming portions to form a self-dimer.

Since the self-dimer primer of the present disclosure is prepared to perform the method of Aspect I according to the present disclosure and be included in the composition of Aspect II according to the present disclosure, the common descriptions between them are omitted in order to avoid undue redundancy leading to the complexity of this specification.

Hereinafter, the present disclosure will be described in more detail with reference to Examples. These Examples are only for the purpose of describing the present disclosure in more detail, and it will be apparent to those skilled in the art to which the present disclosure pertains that the scope of the present disclosure is not limited by these Examples according to the gist of the present disclosure.

### MODE FOR INVENTION

### Examples

### Example 1: Design of Self-Dimer Primer

The self-dimer primer was designed such that its 5′-hairpin-non-forming portion was substantially non-complementary to the entire sequence of another self-dimer primer and its 3′-hairpin-forming portion was substantially complementary to the 3′-hairpin-forming portion of another self-dimer primer.

In particular, in order to prevent the self-dimer primer from forming a hairpin dimer by intra-strand hybridization and to allow the self-dimer primers to form a self-dimer by inter-strand hybridization (i.e., hybridization between 3′-hairpin-forming portions) at 72° C., which is the optimum temperature of activity of Taq DNA polymerase, the 3′-hairpin-forming portion of the self-dimer primer was designed to have a palindrome sequence, and the double-stranded portion of the self-dimer was designed to have a melting temperature (Tm) of 72° C. or higher, while the double-stranded portion of the hairpin dimer was designed to have a Tm much lower than 72° C.

The analysis of the structure and Tm of the self-dimer primer were performed by OligoAnalyzer™ Tool (Integrated DNA Technologies).

If the self-dimer primer has the 5′-hairpin-non-forming portion that is too short compared to the 3′-hairpin-forming portion, the increase in fluorescence signal due to the extension of the self-dimer primers is not significant, making it difficult to analyze the relationship between polymerase activity and fluorescence signal. Therefore, it is preferable to design the 5′-hairpin-non-forming portion of the self-dimer primer to be long to some extent. In consideration of the synthesis yield of the oligonucleotide, the total length of the self-dimer primer was designed to be within 70 mer.

The information on the self-dimer primer as designed is shown in Table 1 below, and the structures of the self-dimer and the hairpin dimer that may be formed by the self-dimer primer are shown in FIG. 3.

TABLE 1

| SEQ | | | | | |
|---|---|---|---|---|---|
| ID | | | Length | *Tm1 | **Tm2 |
| NO | Name | Sequence (5′ to 3′) | (nt) | (°C) | (°C) |
| 1 | Self-Dimer | TCGACCTGCAGGCATGCAAGCTTGGCACGTAA | 60 | 51.5 | 72.1 |
| | Primer | AACGACGGCCAGCTGGCCGTCGTTTTAC | | | |
| | #1 | | | | |

Underlined: 3′-hairpin-forming portion
*Tm1: melting temperature of hairpin dimer
**Tm2: melting temperature of self-dimer

Self-Dimer Primer #1 as designed can form a self-dimer having a Tm of 72.1° C. by inter-strand hybridization between the 3′-hairpin-forming portions of two self-dimer primers, as shown in the top of FIG. 3 as well as a hairpin dimer having a Tm of 51.5° C. by intra-strand hybridization of one self-dimer primer, as shown in the bottom of FIG. 3.

However, formation of the hairpin dimer was expected to be prevented by adjusting the incubation temperature to a temperature higher than 51.5° C.

### Example 2: Preparation of Composition for Analyzing Activity

Self-Dimer Primer #1 as designed in Example 1, 10× ThermoPol buffer (NEB, B9004S), and ultrapure water were mixed in a volume ratio of 10:1:9, heated at 95° C. for 10 minutes, and then cooled to room temperature for 20 minutes to prepare 1 pmol/μL of self-dimer primer reagent.

Then, a final 10 μL composition for analyzing activity was prepared, including 4 pmol of the self-dimer primer reagent, 0.1 mM of deoxynucleotide (dNTP) solution mix, 1.25 M of ROX passive reference dye (BIOTIUM, 29052), 2× EvaGreen (BIOTIUM, 31019), 1× ThermoPol buffer, and ultrapure water.

### Example 3: Measurement of Activity of Taq DNA Polymerase

In order to investigate whether the composition for analyzing activity prepared in Example 2 can be used to measure the activity of a nucleic acid polymerase, a commercially available Taq DNA polymerase (M0267S, NEB) was analyzed.

5 U/μL of the Taq DNA polymerase was diluted with 10× ThermoPol buffer and ultrapure water to concentrations of 62.5 mU/μL, 31.3 mU/μL, 15.6 mU/μL, 7.8 mU/μL, 3.9 mU/μL, 2 mU/μL, and 1 mU/μL, respectively.

Thereafter, the composition for analyzing activity prepared in Example 2, Taq DNA polymerase at each concentration, and ultrapure water were mixed at a volume ratio of 10:1:9. All procedures were carried out on ice.

L of each of the mixtures was placed in an 8-strip PCR tube and incubated at 72° C. in a CFX96 thermocycler (Bio-Rad) capable of measuring fluorescence in real time and controlling temperature. Fluorescence signals were measured at intervals of 30 sec during the incubation.

A plot of fluorescence signal versus time for Taq DNA polymerase at various concentrations is shown in FIG. 4.

As shown in FIG. 4, the fluorescence signal gradually increased over time and finally reached a plateau phase. This indicates that, at the beginning of incubation, Taq DNA polymerase consumes the substrate dNTP and extends the self-dimer to induce an increase in fluorescence signal, and when the dNTP is completely consumed, the fluorescence signal no longer increases. Thus, the change in the fluorescence signal over time will indicate the amount of dNTP consumed in the reaction.

According to the method of the present disclosure, the self-dimer is extended to form an extended dimer into which EvaGreen dyes are intercalated to produce a plot of fluorescence signal versus time as shown in FIG. 4. In the plot, an early phase where the depletion of dNTP does not occur represents a linear function, i.e., a first-order function, and a derivative thereof corresponds to the rate of signal change. As a maximum fluorescence signal is generated upon depletion of the dNTPs added to the reaction, the rate of increase in fluorescence signal can be converted into the consumption of dNTP. For example, dNTPs consumption for a specific time period, e.g., 30 minutes, per a given polymerase concentration (polymerase unit) can be calculated.

The consumption of dNTP for 30 minutes per Taq DNA polymerase concentration as determined above is shown in Table 2 below.

TABLE 2

| Polymerase concentration | dNTP consumption |
|---|---|
| (mU) | (pmol) |
| 62.50 | 249.82 |
| 31.25 | 134.09 |
| 15.63 | 71.54 |
| 7.81 | 40.03 |
| 3.91 | 21.44 |
| 1.95 | 11.33 |
| 0.98 | 6.20 |
| 0.00 | 0.00 |

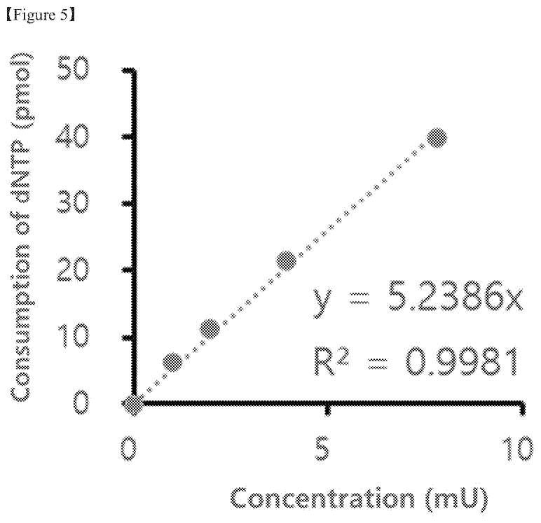

Based on the results in Table 2, the consumption of dNTP per the concentration of Taq DNA polymerase was schematically illustrated in FIG. 5.

As shown in FIG. 5, since the consumption of dNTP versus the concentration of Taq DNA polymerase represents a linear function, the consumption of dNTP in 30 minutes per the unit concentration of Taq DNA polymerase could be confirmed by the slope of the function.

The unit concentration of Taq DNA polymerase used in this Example is equivalent to the unit of Taq DNA polymerase as determined by analysis using radioisotopes (an enzyme unit that consumes 15 nmol of dNTP in 30 minutes at 75° C. as measured by radioisotopes). In FIG. 5, 1 unit of Taq DNA polymerase by analysis using radioisotopes corresponds to a consumption amount of dNTP of 5.24 nmol in 30 minutes at 72° C. according to an embodiment of the method of the present disclosure.

The result shows that the commercially available Taq DNA polymerase has an activity of consuming 5.24 nmol of dNTP in 30 minutes at 72° C. by a method according to an embodiment of the present disclosure.

### Example 4: Application of Oligonucleotides of Various Sequences

To investigate that the method of the present disclosure can be carried out using self-dimer primers of various sequences, 10 μL of compositions for analyzing activity were prepared, comprising each of Self-Dimer Primers #2 to #4 (SEQ ID NOs: 2 to 4) listed in Table 3 below, instead of Self-Dimer Primer #1 of Example 2.

The information on Self-Dimer Primers #2 to 4 is shown in Table 3 below, and the structures of the self-dimers and the hairpin dimers that may be formed by each of Self-Dimer Primers #2 to #4 are shown in FIGS. 6 to 8.

TABLE 3

| SEQ | | | | | |
|---|---|---|---|---|---|
| ID | | | Length | *Tm1 | **Tm2 |
| NO | Name | Sequence (5′ to 3′) | (nt) | (°C) | (°C) |
| 2 | Self- | CTTGGCACGTAAAACGACGGCCAGCTGGCCGTCGTT | 40 | 51.5 | 72.1 |
| | Dimer | TTAC | | | |
| | Primer #2 | | | | |
| 3 | Self- | CATGCAAGCTTGGCACGTAAAACGACGGCCAGCTG | 48 | 51.5 | 72.1 |
| | Dimer | GCCGTCGTTTTAC | | | |
| | Primer #3 | | | | |
| 4 | Self- | TCAATCACCAGCCTAGCTACGTGTAAGCTCGGCCGA | 48 | 46.3 | 70.5 |
| | Dimer | GCTTACACGTAG | | | |
| | Primer #4 | | | | |

Underlined: 3′-hairpin-forming portion
*Tm1: melting temperature of hairpin dimer
**Tm2: melting temperature of self-dimer

Each of the compositions for analyzing activity, Taq DNA polymerases (NEB, M0267S) at various concentrations, prepared in Example 3, and ultrapure water were mixed at a volume ratio of 10:1:9. All procedures were carried out on ice.

20 μL of each of the above mixtures was placed in an 8-strip PCR tube and then incubated in a CFX96 thermocycler (Bio-Rad) at 72° C. Fluorescence signals were measured at intervals of 30 sec during the incubation.

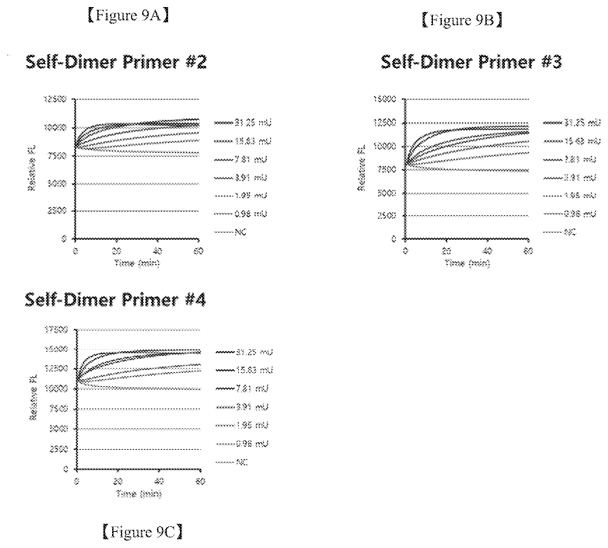

The measurement results are shown in FIGS. 9A-9C. FIG. 9A shows a plot of fluorescence signal versus time, which is obtained using Self-Dimer Primer #2; FIG. 9B shows a plot of fluorescence signal versus time, which is obtained using Self-Dimer Primer #3; FIG. 9C shows a plot of fluorescence signal versus time, which is obtained using Self-Dimer Primer #4.

The results demonstrate that the activity of polymerase can be measured by using self-dimer primers having various sequences.

Furthermore, comparing the results of FIGS. 9A-9C with those of FIG. 4 in Example 3, it can be seen that Self-Dimer Primer #1 exhibits a higher change in fluorescence signal in a unit time than Self-Dimer Primers #2 to #4, indicating that Self-Dimer Primer #1 has a higher sensitivity. This is probably due to the longer oligonucleotide length and in particular the longer 5′-hairpin-free portion of Self-Dimer Primer #1 compared to Self-Dimer Primers #2 to #4.

### Example 5: Measurement of Activity of Various Polymerases

In addition to the Taq DNA polymerase tested in Example 3, the activities of various types of polymerases used in the diagnostic field were measured.

Commercially available Taq DNA polymerase (Roche, 11147633103) and rTth DNA polymerase (TOYOBO, TTH-301) were diluted to concentrations of 62.5 mU/μL, 31.3 mU/μL, 15.6 mU/μL, 7.8 mU/μL, 3.9 mU/μL, 2 mU/μL, and 1 mU/μL, respectively. In addition, commercially available Bst DNA polymerase, large fragment (NEB, M075S) was diluted to concentrations of 10 mU/μL, 5 mU/μL, 2.5 mU/μL, 1.25 mU/μL, 0.63 mU/μL, and 0.31 mU/μL, and Pfu DNA polymerase (PROMEGA, M7741) was diluted to concentrations of 250 mU/μL, 125 mU/μL, 62.5 mU/μL, 31.3 mU/μL, 15.6 mU/μL, 7.8 mU/μL, and 3.9 mU/μL.

Thereafter, the composition for analyzing activity prepared in Example 2, each of the polymerases at various concentrations, and ultrapure water were mixed at a volume ratio of 10:1:9. All procedures were carried out on ice.

Then, 20 μL of each of the mixtures was placed in an 8-strip PCR tube, and then incubated in a CFX96 thermocycler (Bio-Rad) at 72° C. (65° C. in the case of Bst DNA polymerase). Fluorescence signals were measured at intervals of 30 sec during the incubation.

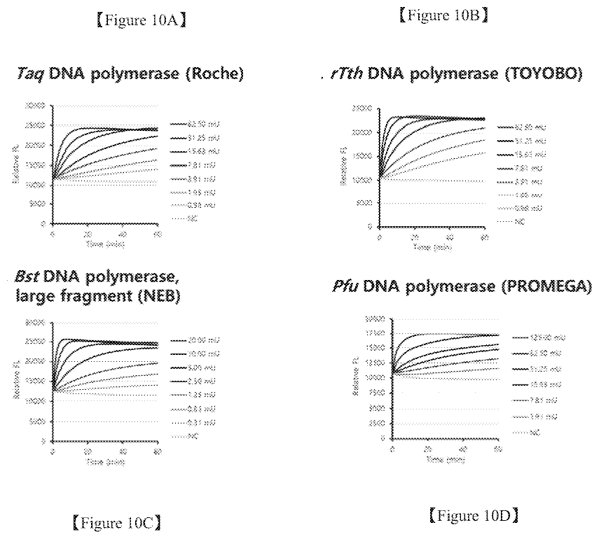

The measurement results are shown in FIGS. 10A-10D. FIG. 10A shows a plot of fluorescence signal versus time, which is obtained using Taq DNA polymerase (Roche); FIG. 10B shows a plot of fluorescence signal versus time, which is obtained using rTth DNA polymerase (TOYOBO); FIG. 10C shows a plot of fluorescence signal versus time, which is obtained using Bst DNA polymerase, large fragment (NEB); FIG. 10D shows a plot of fluorescence signal versus time, which is obtained using Pfu DNA polymerase (PROMEGA).

Then, as described in Example 3, the consumption of the dNTP in 30 minutes per the polymerase concentration was calculated.

The results are shown in Table 4 below.

TABLE 4

| Taq DNA polymerase (Roche) | | rTth DNA polymerase (TOYOBO) | |
|---|---|---|---|
| Enzyme | Consumption | Enzyme | Consumption |
| concentration | of dNTP | concentration | of dNTP |
| (mU) | (pmol) | (mU) | (pmol) |
| 62.50 | 176.98 | 62.50 | 308.76 |
| 31.25 | 87.46 | 31.25 | 180.53 |
| 15.63 | 50.96 | 15.63 | 99.38 |
| 7.81 | 29.65 | 7.81 | 56.12 |
| 3.91 | 15.70 | 3.91 | 29.27 |
| 1.95 | 7.64 | 1.95 | 13.31 |
| 0.98 | 3.41 | 0.98 | 7.45 |
| 0.00 | 0.00 | 0.00 | 0.00 |

| Bst DNA polymerase (NEB) | | Pfu DNA polymerase (PROMEGA) | |
|---|---|---|---|
| Enzyme | Consumption | Enzyme | Consumption |
| concentration | of dNTP | concentration | of dNTP |
| (mU) | (pmol) | (mU) | (pmol) |
| 10.00 | 136.69 | 125.00 | 144.96 |
| 5.00 | 77.54 | 62.50 | 71.40 |
| 2.50 | 45.08 | 31.25 | 33.42 |
| 1.25 | 19.12 | 15.63 | 17.88 |

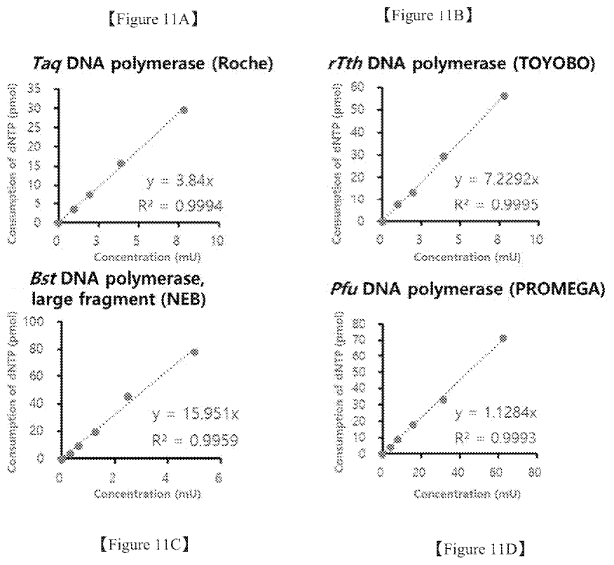

In addition, based on the results in Table 4, the consumption of dNTP per the concentration of polymerase was calculated and depicted in FIGS. 11A-11D.

As shown in FIGS. 11A-11D, it was confirmed that the definition of 1 unit based on radioisotope measurement of each polymerase (consuming 10 nmol of dNTP in 30 min at 75° C.; in case of Bst polymerase, consuming 10 nmol of dNTP in 30 minutes at 65° C.) corresponded to levels of consuming 3.84 nmol of dNTP in 30 minutes at 72° C. for Taq DNA polymerase (Roche), 7.23 nmol of dNTP in 30 minutes at 72° C. for rTth DNA polymerase, 15.95 nmol of dNTP in 30 minutes at 65° C. for Bst DNA polymerase, and 1.13 nmol of dNTP in 30 minutes at 72° C. for Pfu DNA polymerase, according to the analysis of activity using the self-dimer primer of the present disclosure.

The results demonstrate that the activity of various polymerases can be measured using the method of the present disclosure.

Furthermore, when comparing the activity of Taq polymerase from Roche in Example 5 and the activity of Taq polymerase from NEB in Example 3, Taq polymerase from Roche consumed 3.84 nmol of dNTP in 30 minutes at 72° C., whereas Taq polymerase from NEB consumed 5.24 nmol of dNTP in 30 minutes at 72° C. Accordingly, it was found that Taq polymerase from NEB had more excellent polymerization activity than that from Roche.

The comparison shows that the activities between polymerases can be compared using the method of the present disclosure.

Having described a preferred embodiment of the present invention, it is to be understood that variants and modifications thereof falling within the spirit of the invention may become apparent to those skilled in this art, and the scope of this invention is to be determined by appended claims and their equivalents.

1. A method for analyzing the activity of a nucleic acid polymerase of interest, comprising the steps of:
(a) incubating the nucleic acid polymerase of interest with a composition for analyzing activity comprising: (i) a self-dimer primer, (ii) an intercalating dye, and (iii) dNTPs or NTPs, wherein the self-dimer primer comprises, in the 5′ to 3′ direction, a 5′-hairpin-non-forming portion and a 3′-hairpin-forming portion, wherein during said incubating, the self-dimer primer hybridizes with another self-dimer primer between 3′-hairpin-forming portions to form a self-dimer, and both strands of the resulting self-dimer are extended by the activity of the nucleic acid polymerase of interest to form an extended dimer, wherein the formation of the extended dimer provides a detectable signal from the intercalating dye; and
(b) measuring the detectable signal over time, wherein the change in signal over time is indicative of the activity of the nucleic acid polymerase of interest.

2. The method of claim 1, wherein the nucleic acid polymerase of interest is a DNA polymerase.

3. The method of claim 2, wherein the DNA polymerase is derived from a bacterium selected from the group consisting of Thermus aquaticus, Thermus thermophilus, Thermus filiformis, Thermus flavus, Thermococcus litoralis, Thermus antranikianii, Thermus caldophilus, Thermus chliarophilus, Thermus igniterrae, Thermus lacteus, Thermus oshimai, Thermus ruber, Thermus rubens, Thermus scotoductus, Thermus silvanus, Thermus species Z05, Thermus species sps 17, Thermotoga maritima, Thermotoga neapolitana, Thermosipho africanus, Thermococcus barossii, Thermococcus gorgonarius, Pyrococcus furiosus, Pyrococcus woesei, Pyrococcus horikoshii, Pyrococcus abyssi, Pyrodictium occultum, Aquifex pyrophilus, and Aquifex aeolicus.

4. The method of claim 1, wherein the 5′-hairpin-non-forming portion of the self-dimer primer has a nucleotide sequence substantially non-complementary to the 5′-hairpin-non-forming portion and the 3′-hairpin-forming portion of another self-dimer primer.

5. The method according to claim 1, wherein the 5′-hairpin-non-forming portion of the self-dimer primer is 5 to 50 nucleotides in length.

6. The method of claim 1, wherein the 3′-hairpin-forming portion of the self-dimer primer has a nucleotide sequence substantially complementary to the 3′-hairpin-forming portion of another self-dimer primer.

7. The method of claim 1, wherein the 3′-hairpin-forming portion of the self-dimer primer has a substantially palindromic sequence.

8. The method according to claim 1, wherein when the 3′-hairpin-forming portion of the self-dimer primer is n nucleotides in length, the m^{th} nucleotide base from the 3′-terminus is complementary to the n-m+1th nucleotide base, wherein n is an integer from 5 to 50, and m is an integer of 1 or greater.

9. The method according to claim 1, wherein the 3′-hairpin-forming portion of the self-dimer primer is 5 to 50 nucleotides in length.

10. The method according to claim 1, wherein the ratio of the length of the 5′-hairpin-non-forming portion to the length of the 3′-hairpin-forming portion of the self-dimer primer is about 1:1.

11. The method of claim 1, wherein the self-dimer primer is 10 to 100 nucleotides in length.

12. The method of claim 1, wherein the intercalating dye is selected from the group consisting of EvaGreen, ethidium bromide, SYBR Green I, SYBR Gold, Yo-Yo, Yo-Pro, TOTO, SYTO9, BEBO, BOXTO, Hoechst dyes, LC Green Plus, ResoLight, Chromofy, and PicoGreen.

13. The method of claim 1, wherein the incubating is performed under isothermal conditions such that the nucleic acid polymerase of interest functions and the self-dimer primer does not form a hairpin dimer by intra-strand hybridization.

14. The method of claim 13, wherein the isothermal condition is at a temperature selected from 65 to 80° C.

15. The method according to claim 1, wherein the composition for analyzing activity does not comprise any additional oligonucleotide other than the self-dimer primer.

16. The method of claim 1, wherein the change in signal is measured over a period of time during which a plot of signal intensity versus incubation time represents a linear function.

17. The method of claim 1, wherein the change in signal over time is converted into an amount of dNTP consumed in a unit time of incubation, thereby indicating the activity of the nucleic acid polymerase of interest.

18. A composition for analyzing the activity of a nucleic acid polymerase of interest, comprising a self-dimer primer,
wherein the self-dimer primer comprises, in the 5′ to 3′ direction, a 5′-hairpin-non-forming portion and a 3′-hairpin-forming portion,
wherein during said incubating, the self-dimer primer hybridizes with another self-dimer primer between 3′-hairpin-forming portions to form a self-dimer, and both strands of the resulting self-dimer are extended by the activity of the nucleic acid polymerase of interest to form an extended dimer, wherein the formation of the extended dimer provides a detectable signal from the intercalating dye,
wherein the change in signal over time is indicative of the activity of the nucleic acid polymerase.

19. The composition of claim 18, further comprising an intercalating dye.

20. The composition of claim 18, further comprising dNTPs or NTPs.

21. A self-dimer primer comprising, in the 5′ to 3′ direction, a 5′-hairpin-non-forming portion and a 3′-hairpin-forming portion,
wherein the 5′-hairpin-non-forming portion has a nucleotide sequence substantially non-complementary to the 5′-hairpin-non-forming portion and the 3′-hairpin-forming portion of another self-dimer primer, and the 3′-hairpin-forming portion has a nucleotide sequence substantially complementary to the 3′-hairpin-forming portion of another self-dimer primer,
wherein the self-dimer primer is capable of hybridizing with another self-dimer primer between 3′-hairpin-forming portions to form a self-dimer.


SEQUENCE LISTING (4 sequences)

SEQ ID NO: 1 - DNA, 60 bp, synthetic construct
  source 1..60: mol_type = other DNA
tcgacctgcaggcatgcaagcttggcacgtaaaacgacggccagctggccgtcgttttac

SEQ ID NO: 2 - DNA, 40 bp, synthetic construct
  source 1..40: mol_type = other DNA
cttggcacgtaaaacgacggccagctggccgtcgttttac

SEQ ID NO: 3 - DNA, 48 bp, synthetic construct
  source 1..48: mol_type = other DNA
catgcaagcttggcacgtaaaacgacggccagctggccgtcgttttac

SEQ ID NO: 4 - DNA, 48 bp, synthetic construct
  source 1..48: mol_type = other DNA
tcaatcaccagcctagctacgtgtaagctcggccgagcttacacgtag